(19) United States
(12) Patent Application Publication
(10) Pub. No.: US 2020/0216910 A1
(43) Pub. Date: Jul. 9, 2020

(54) METHOD AND SYSTEM FOR ANALYSIS OF DNA METHYLATION AND USE OF SAME TO DETECT CANCER

(71) Applicant: ENRICH BIOSCIENCE INC., Etobicoke (CA)

(72) Inventor: Tarang KHARE, Etobicoke (CA)

(21) Appl. No.: 16/637,552

(22) PCT Filed: Aug. 9, 2018
(86) PCT No.: PCT/CA2018/050966

§ 371 Date: Feb. 7, 2020

Related U.S. Application Data
(60) Provisional application No. 62/542,926, filed on Aug. 9, 2017.

(51) Int. Cl. C12Q 1/6886; C12Q 1/6806

(52) U.S. Cl. CPC C12Q 1/6886; C12Q 2600/154; C12Q 1/6806

(57) ABSTRACT

Methods for detecting and analyzing low abundance and fragmented nucleic acids are provided, for example for amplifying and analyzing cancer cell DNA having a known pattern of DNA methylation. The example method includes a linear amplification step for targeting an area of interest and creating a complementary strand of the particular area of interest. In an example method a

### FIELD

The present disclosure relates generally to a system and method for DNA analysis and particularly to a method and system for the analysis of DNA using methylation biomarkers, for example in cancer cells.

### BACKGROUND

Cancer is a multifactorial and a polygenic disorder involving multiple pathways. Additionally, cancer is heterogeneous, meaning that tumor cells can show distinct morphological and phenotypic profiles from one another. This complicates the cancer detection method when performed on relatively few biomarkers. Research has shown that the involved genes either subjected to higher sequence mutation or acquire DNA methylation at the gene promoter and thereby, the associated gene is non-functional (Markowitz, Sanford D., and Monica M. Bertagnolli. 2009. “Molecular Basis of Colorectal Cancer.” New England Journal of Medicine 361 (25): 2449-60. doi:10.1056/NEJMra0804588, incorporated herein by reference).

Further, there is a possibility that promoter methylation is alleviated. In this regard, normally inactive genes have an ectopic expression resulting in an abnormal phenotype. Other genomic regions, such as intergenic, intragenic or genic regions may also have differential DNA methylation that is directly or indirectly involved in the cancer progression. These regions are also candidate biomarkers for cancer detection.

When cancer progresses it becomes highly vascularized and cancer cells and fragmented DNA tend to be shed from apoptotic cells into the bloodstream.

The proportion of cancer DNA is small compared to the blood DNA. It is challenging to differentiate the cancer DNA from the normal cell DNA in non-invasive samples.

Epigenetic tools including tools for DNA modification can be applied to differentiate between cancer cells and normal cells, and can also be applied to capture cancer DNA that has higher or lower methylation levels compared to a normal cell at a locus. The capture of such differentially methylated regions will form a signature specific to a cancer type or subtype.

Epigenetic modifications, DNA methylation and histone modification, are examples of changes in gene expression and cellular phenotype without corresponding changes in the DNA sequence. For example, an important epigenetic mechanism for silencing tumor suppressor genes (TSG) during carcinogenesis is by hypermethylating TSG promoters (Esteller, Manel. 2002. “CpG Island Hypermethylation and Tumor Suppressor Genes: A Booming Present, a Brighter Future.” Oncogene 21 (35): 5427-40. doi:10.1038/sj.onc.1205600; Hoon, Dave S B, Mia Spugnardi, Christine Kuo, Sharon K Huang, Donald L Morton, and Bret Taback. 2004. “Profiling Epigenetic Inactivation of Tumor Suppressor Genes in Tumors and Plasma from Cutaneous Melanoma Patients.” Oncogene 23 (22): 4014-22. doi:10.1038/sj.onc.1207505, both incorporated herein by reference).

DNA Methylation is a naturally occurring epigenetic modification on human DNA, where a methyl group is covalently attached to a cytosine base, preferentially at CpG sites, also known as CG sites. CpG sites are sites within a DNA strand where, in the 5′43′ direction, a cytosine is followed by a guanine (in other words, in common DNA notation, 5′-cytosine-phosphate-guanine-3′).

DNA methylation on cytosine is a significant known DNA modification in mammals. Mammalian gene promoters are often associated with CpG rich (CpG island) regions and are unmodified at all stages of development and tissue types (Jones, Peter A. 2012. “Functions of DNA Methylation: Islands, Start Sites, Gene Bodies and beyond.” Nature Reviews Genetics 13 (7): 484-92. doi:10.1038/nrg3230, incorporated herein by reference). When the gene promoter is methylated, the associated gene is stably silenced. Approximately seventy percent of genes harbor high density of CpG dinucleotides—CpG islands—in their promoter, but only about 5% of these are methylated in normal cells illustrating that the establishment of this epigenetic mark is not a predominant process.

De novo DNA methylation is rare in adult somatic tissues and is mostly observed during differentiation, ageing and in cancer cells. In cancer cells de novo DNA methylation is at the TSG promoter and this DNA modification makes cells epigenetically distinct from the normal cell DNA. Additionally, different cancer origin shows the silencing of different TSG genes providing a unique signature. Asymptomatic DNA methylation (non-CpG), as well as other oxidative forms of DNA modifications such as 5-hydroxymethyl, 5-formyl, and 5-carboxyl-cytosine are also present in the normal cell, but they are in minor proportion when compared to the 5-methyl-cytosine. Moreover, 5-methyl-cytosines play the major role in silencing of the associated gene. DNA methylation pattern analysis holds great potential for the various applications, ranging from the disease (cancer) progression, monitoring, diagnosis, therapy and in research.

A known method for analysis of the extent of DNA methylation in a sample of genomic DNA is bisulfite (BS) chemical treatment, which converts unmodified cytosine residues to uracil, whereas modified (DNA methylated) cytosines and other nucleotides remain unchanged (FIG. 1). The bisulfite converted DNA can then be subjected to various downstream applications, including, PCR amplification of a single locus, whole genome sequencing, and/or 450K bead array hybridization, for profiling and estimating the amount of DNA methylation in the DNA sample.

The DNA methylation from various genomic regions forms a cell type specific pattern, which plays an important role during cell development and cell differentiation. In cancer cells, this pattern is skewed and a totally new and cancer-specific pattern is formed. This pattern may further change during the cancer progression and treatment. Thus, the DNA methylation pattern can be used to diagnose cancer, estimate progression of the cancer, and track the effectiveness of treatment.

Growing cancer cells naturally shed DNA called circulating cell free DNA (cfDNA; for tumor DNA also called as ctDNA) into the body fluids such as blood. Cancer-specific DNA methylation patterns can be found in the detached tumor cells in body fluids. The methylation patterns correlate with DNA methylation patterns of the tissue biopsies. The cancer-specific DNA methylation pattern can be detected by analyzing cancer cells and cfDNA present in the bloodstream (Warton, Kristina, and Goli Samimi. 2015. “Methylation of Cell-Free Circulating DNA in the Diagnosis of Cancer.” Frontiers in Molecular Biosciences 2. doi:10.3389/fmolb.2015.00013, incorporated herein by reference).

It would be beneficial to collect and analyze the cancer DNA present in the bloodstream for effective treatment and analysis. A challenge in the analysis and amplification process is that the cfDNA is fragmented (size range 100-500bp) (Volik, Stanislav, Miguel Alcaide, Ryan D Morin, and Colin Collins. 2016. “Cell-Free DNA (cfDNA): Clinical Significance and Utility in Cancer Shaped By Emerging Technologies.” Molecular Cancer Research 14 (10): 898 LP-908. http://mcr.aacrjournals.org/content/14/10/898.abstract, incorporated herein by reference), and when harsh bisulfite chemical treatment is applied for DNA methylation analysis, these fragments are degraded to a greater extent.

### SUMMARY OF THE INVENTION

Example embodiments disclosed herein are methods for detecting low abundance and fragmented nucleic acids, and in particular, determining the level of cytosine methylation of said low abundance and fragmented nucleic acids. In particular, the example method includes a linear pre-amplification step for targeting an area of interest and creating a complementary strand of a particular area of interest. In an example method a Multiplex Polymerase Chain Reaction (Multiplex PCR) is implemented after the linear amplification step.

According to certain embodiments of the present invention is provided a method for DNA analysis comprising: (a) chemically treating genomic DNA via bisulfite treatment and converting cytosine residues to uracil residues; (b) linearly amplifying the chemically treated genomic DNA and generating a complimentary template to the genomic DNA; and (c) amplifying the complementary template via multiplex polymerase chain reaction (PCR). In certain embodiments, the amplified complimentary template can be analyzed to determine the extent and/or pattern of cytosine methylation in the genomic DNA.

In one example aspect there is provided a method for analysis of a sample nucleic acid sequence containing methylated cytosine, comprising: providing a sample of nucleic acid sequences; chemical treatment of said sample nucleic acid sequences resulting in a conversion of unmethylated cytosine residues in said sample nucleic acid sequence to uracil; linearly amplifying said chemically treated nucleic acid sequence to generate a complimentary template to the chemically treated nucleic acid sequence; and amplifying the complementary template via multiplex polymerase chain reaction (PCR) to generate a library of amplified nucleic acid sequences; wherein the library of amplified nucleic acid sequences preferentially contain sequences from the sample nucleic acid that contained methylated cytosine.

In one example aspect, there is provided a method for analysis of a sample nucleic acid sequence containing methylated cytosine, comprising: providing a sample of nucleic acid sequences; chemical treatment of said sample nucleic acid sequences resulting in a conversion of unmethylated cytosine residues in said sample nucleic acid sequence to uracil; linearly amplifying said chemically treated nucleic acid sequence to generate a complimentary template to the chemically treated nucleic acid sequence; contacting the sample with a plurality of nucleic acid probes, wherein the probes are designed to hybridize randomly along a target nucleic acid sequence; allowing hybridization of the plurality of nucleic acid probes to the target nucleic acid sequence; forming a plurality of circular nucleic acid sequences, each of the circular sequences comprising a nucleic acid probe sequence and a target nucleic acid sequence; amplifying the plurality of circular nucleic acid sequences to form a plurality of amplified target nucleic acid sequences; and optionally, sequencing the amplified target nucleic acid sequences, wherein the plurality of amplified nucleic acid sequences preferentially contain sequences from the sample nucleic acid that contained methylated cytosine.

In a further example aspect, the chemically treated nucleic acid sequence is cleaved at uracil residues utilizing a uracil DNA glycosylase enzyme, after the linear amplification step.

In a further example aspect, the sample nucleic acid sequence is genomic DNA, preferably whole genomic DNA.

In a further example aspect, the genomic DNA is isolated from a blood sample from a patient.

In a further example aspect, at least one primer is used to target and overlap a region of interest of the genomic nucleic acid sequence during the linear amplification step.

In a further example aspect, the at least one primer comprises a CpG dinucleotide for preferential amplification of methylated fragments of the region of interest.

In a further example aspect, the at least one primer comprises a TpG, where T is a converted C, nucleotide for preferential amplification of unmethylated fragments of the region of interest.

In a further example aspect, at least two primers are used during the multiplex PCR step.

In a further example aspect, the multiplex PCR comprises two to twenty-two cycles.

In a further example aspect, the conversion of cytosine residues further comprises converting unmethylated cytosine residues, wherein 5-methyl-cytosine residues remain unchanged.

In a further example aspect, the chemical treatment is a bisulfite treatment.

In a further example aspect, the probes are designed to hybridize to promoter regions along a target nucleic acid sequence.

In a further example aspect, amplification primers hybridize to nucleic acid probe sequences during the multiplex amplification step.

In a further example aspect, the nucleic acid probes are padlock probes.

In a further example aspect, the target nucleic acid sequence is a gene or a promoter region or an intergenic region.

In a further example there is provided a repair step wherein DNA fragments are annealed together, prior to the bisulfite conversion.

In a further example there is provided with a genomic region capture step, prior to the multiplex amplification step.

In a further embodiment there is provided with a template improvement step following the multiplex amplification step. In one example, said template improvement step comprises amplification with the phi29 polymerase.

In a further embodiment, there is provided a method of determining whether a patient has cancer, comprising performing the method of any one of the preceding claims to a sample from the patient, and comparing the amount of amplified DNA from the method to a control sample, wherein a higher amount of amplified DNA is determinative of cancer.

In yet a further embodiment, the sample is a blood sample.

### BRIEF DESCRIPTION OF THE DRAWINGS

Reference will now be made, by way of example, to the accompanying drawings which show example embodiments of the present application, and in which:

FIG. 1 is an example flowchart of a bisulfate conversion of example genomic DNA;

FIG. 2 is an example flowchart of an example embodiment of the process of the present invention;

FIG. 3 is an example schematic image of probes and primers of an example embodiment;

FIG. 4 is an example flowchart of an example embodiment of a portion of the process of the present invention;

FIG. 5 is an example flowchart of the continuation of FIG. 4;

FIG. 6 is an example graph of Ct values compared to a number of amplification over 0-30 cycles at four different loci;

FIG. 7 is an example chart of Loge fold change over 0-30 cycles at four different loci;

FIG. 8 is an example chart of the influence of annealing time at 1 hour, 2 hours, and 8 hours on amplification at five different loci;

FIG. 9 is an example chart of fold difference from 8 hours annealing time at five different loci;

FIG. 10 is an example of size separation electrophoresis of locus specific amplified DNA at two different loci from templates generated after 1 hour, 2 hours and 8 hours annealing incubation time;

FIG. 11 is an example graph for detecting a fraction of methylated DNA as fold change from the reference 100% methylated control human DNA;

FIG. 12 is an example chart of raw Ct values obtained from the different concentration of methylated DNA for an example of the traditional method;

FIG. 13 is an example graph of the chart data of FIG. 11 as fold change from the reference 100% methylated DNA for an example traditional method;

FIG. 14 is an example chart of raw Ct values obtained from the different concentration of methylated DNA for an example method of the present invention (Enrich method);

FIG. 15 is an example graph of the chart data of FIG. 11 of fold change from the reference 2.5% methylated DNA for an example of the present invention (Enrich method);

FIG. 16 shows PCR products with and without the template improvement step;

FIG. 17 shows the fragment size of DNA from tumor samples;

FIG. 18 shows a melt curve analysis of samples post-method;

FIG. 19 shows an example melt curve analysis showing end-point PCR product of RASS1Fa probes post-method;

FIG. 20 shows the testing of locus-specific end-point amplification for various biomarkers on colorectal cancer case-samples, post-method.

Similar reference numerals may have been used in different figures to denote similar components.

### DETAILED DESCRIPTION

The present disclosure provides an example method for detecting and amplifying known DNA methylation at cytosine sites that constitute a disease-specific pattern.

The example method including a linear amplification step (in other words, a pre-amplification step) allows multiplex probes to efficiently and accurately amplify the region of interest by creating a complementary strand of a particular region of interest. The linear amplified templates provide a higher probability for the probes to anneal to the region of interest.

The present example method is disclosed using cancer DNA as an example. However, the example method is applicable to a variety of other disorders including any disorder concerning the investigation of multiple genomic regions on a fragmented or native DNA.

The present example method may be used for preferentially amplifying methylated DNA regions, in particular, those of cancer cells, which are different from normal cells and from other inflammatory or diseased conditions. However, this example method can be applied to detect any other molecular entity including DNA sequence mutations, RNA transcript or miRNA, with some modifications of the probe design.

The present example method can also be used for amplifying unmethylated regions of cancer DNA compared to the methylated locus of a normal cell.

### Example Method

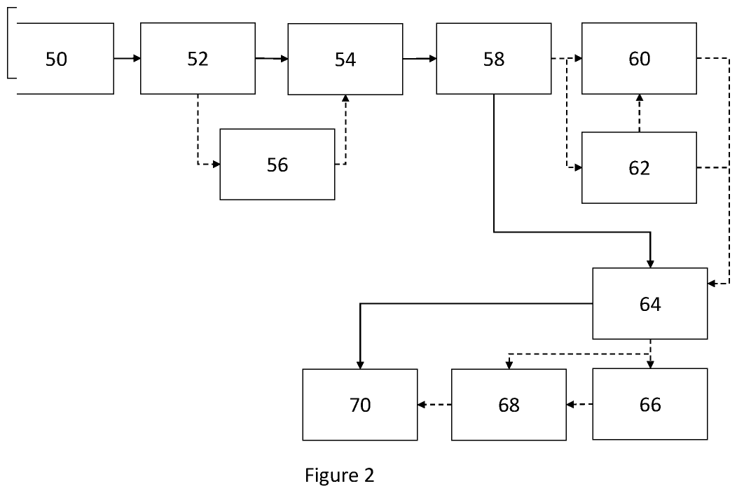

An example method is provided (the “Enrich” example method), constituting the following steps set forth below, and as disclosed in the flow chart of FIG. 2.

### Step 1: Genomic DNA (gDNA) Collection 50 and Purification 52

gDNA to be assayed can be obtained from a variety of sources, for example, from a human or an animal, and for example, from body fluids. Example body fluids include blood, plasma, urine, stool, sputum or a biopsy sample from the affected tissue. Preferentially, the body fluid is blood fluid containing cell free circulating DNA (cfDNA).

In one example, blood fluid is collected from a human. The first step of the method is this DNA obtaining step 50.

Genomic DNA was purified 52 from the blood fluid by known methods, for example, the ZymoBead™ Genomic DNA kit (Zymo Research Corp, Irvine Calif.). Alternatively, total DNA (genomic, viral, cfDNA, and mitochondrial) was purified from the whole blood utilizing known methods, such as the QIAamp DNA Blood Mini Kit (Qiagen NV, The Netherlands) or the NucleoSpin DNA purification method (Macherey-Nagel), utilizing standard instructions and steps.

### Step 2: Bisulfite Conversion Step 54

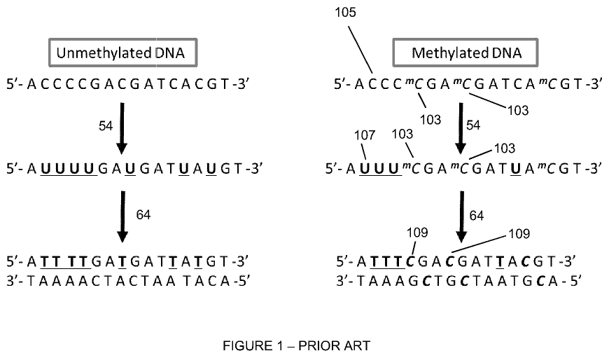

The purified DNA was then subjected to a bisulfite conversion step 54, as previously taught in the art. With reference to FIG. 1, which explains, in an example, the bisulfite conversion step 54, and its effect on both methylated and unmethylated DNA, bisulfite chemical treatment of the genomic DNA converts unmethylated cytosine residues 105 to uracil residues 107 while leaving any 5-methyl-cytosine (^{m}C) residues 103 unchanged. This forms the basis for identifying methylated cytosines 103.

The result of the bisulfite conversion step is DNA containing uracil 107 where non-methylated cytosines 105 were found in the gDNA. Therefore, any cytosines found in the BS DNA will be known to be methylated.

### Step 2A: Optional Repair Step 56

Optionally, before the bisulfite conversion step 54, the isolated gDNA can be ligated together to form longer templates. gDNA ends are repaired, then ligated, using known techniques. These longer templates are found to have less degradation during bisulfite conversion step 54, in comparison to non-ligated fragmented DNA.

### Step 3: Linear Amplification 58

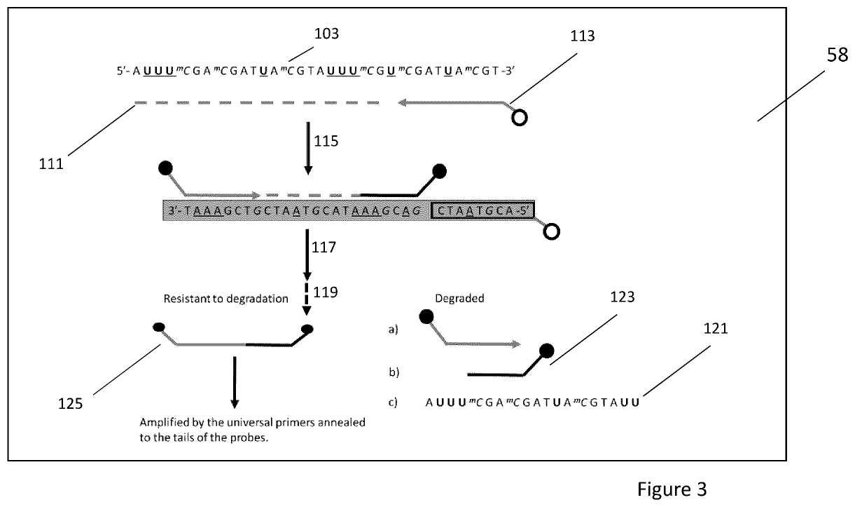

Linear amplification is described with reference to FIGS. 1 and 3.

The linear amplification step 58 comprises a targeted and methylation-specific linear amplification of the bisulfite converted DNA from step 2. Methylated region 103 of DNA is preferentially and/or selectively amplified, as disclosed in FIGS. 1 and 3, since it has not been converted to uracil in the bisulfite conversion step. In certain preferred embodiments, and as shown, the primers used to target and overlap the methylated group.

For targeted and methylation-specific linear amplification, region specific single primers are utilized; in certain embodiments, the primers include “CpG” dinucleotide within their sequence. This allows for preferential amplification of the methylated fragments of a region of interest, since only the methylated fragments will contain cytosine. Alternatively, in other embodiments, the primers can include “TpG”, where T is a bisulfite converted C, dinucleotide within their sequence, for preferential amplification of unmethylated fragments of a region of interest.

Linear amplification 58 allows for the generation of relatively long templates for the example downstream multiplex probe step disclosed below. Further, linear amplification 58 provides a relatively unbiased linear amplification of different regions since it not an exponential amplification, where multiple primer-pairs competes for the available resources. Finally, it is believed that linear amplification 58 can minimize false amplification during multiplexing on a bisulfite converted DNA. This is due to the concept that, while double stranded DNA has two complementary strands (with Adenine (A) complementing with Thymine (T) and cytosine (C) complimenting with guanine (G)), the bisulfite converted genomic DNA comprises DNA where all unmethylated Cytosines have been converted to uracil—which complements with adenine rather than guanine—therefore, bisulfite treated genome comprises mostly of the three base composition (A, G, T) and with minimal cytosine. This bisulfite converted genome suffers from an inherent issue that it increases the probability of primers/probes for ectopic annealing and amplification of the wrong target regions.

The linear amplification step generates a complimentary template 111. Since, the multiplex probes of the present invention and bisulfite converted DNA share a similar composition of nucleotides, i.e. a lower percentage of cytosine in the DNA, and therefore, multiplex probes of the present invention have a lower probability of ectopic annealing to the bisulfite converted simplified whole genome, and thereby, reduce the false positive rate.

It is noted that the primer 113 used in this linear amplification step is preferably different than the primers used in the multiplex PCR step (described below). This is believed to improve further the specificity of the region of interest that is amplified. This step is similar to the principles of the prior art of semi-nested or nested PCR for improving the specificity of the amplified target region.

The step 58 utilizes a linear amplification primer 113, and a known linear amplification methodology. The linear amplified product is now a template for a primer pair (123a and 123b), where both primers anneal to the same template strand, and one primer extends (123a) and ligates to the other (123b).

The reaction is later subjected to an exonuclease step 117, which degrades all bisulfite converted DNA miss-targeted linearly amplified fragments 121 as well as the unutilized/non-annealed probes 123, leaving the amplifiable fragments 125, which is resistant -resistance is represented as solid black circle on both ends (5′ and 3′) of the fragment—from being cleaved by the exonuclease enzymes. These fragments are exponentially PCR amplified by the universal primer—pair, which anneal to the tails of the probes.

Optionally, the exonuclease step 117 can be followed by an optional USER step 119, where non-specific amplification can be further suppressed by the usage of uracil DNA glycosylase enzyme (USER), which specifically cleaves any uracil in the template or in the PCR product. The bisulfite converted DNA has uracil residue resulting from the unmethylated cytosine on the bisulfite treatment. This uracil after linear amplification or the after multiplex step will convert to T, and thereby, the newly generated template is resistant to the USER cleavage. Although in this Example, the USER step 119 is shown as an optional element after the linear amplification step 58, but it may also be beneficial after the linear extension and ligation step of 64 and before the exponential, with a pair of universal primers, PCR step of 64.

It has been found that the use of a linear amplification step 58 “cleans” and greatly enhances the starting material for the traditional PCR/multiplex DNA amplification step which follows.

### Step 3A: Optional Cleaning Step 60

In certain embodiments, the solution containing the linearly amplified fragments 125 resulting from the linear amplification step 58 is cleaned in a cleaning step 60. The cleaning can be through any known means, including one or more of treatment such as with Shrimp Alkaline Phosphatase (rSAP) which dephosphorylates the remaining dNTP, a substrate for the DNA polymerase enzyme activity; a pre-PCR clean up using a column that can retain single stranded DNA (such as a commercially available nucleotide removal column from Qiagen); or use of biotinylated primers and a streptavidin beads purification step. Pre-PCR cleaning of DNA, to improve the signal/noise ratio of the PCR amplification, is generally known.

### Step 3B: Optional Genomic Region Capture Step 62

A further optional step, to capture genomic regions, is to anneal the linearly amplified fragments generated by primers 113 to the padlock probes, followed by extension and ligation of the annealed DNA; the ligated products form single stranded circles that are resistant to the exonucleases. The samples were then subjected to exonuclease, to degrade the non-circularized DNA. This genomic region capture step results in a sample that is much more enriched in genomic DNA of the region of interest.

Alternatively, the cleaning step 60 can be done after the genomic region capture step 62, or a second cleaning step can be done before the PCR amplification.

### Step 4: Multiplex Polymerase Chain Reaction (PCR) Step 64.

In the following example step, Multiplex polymerase chain reaction (Multiplex PCR) is used to amplify several different DNA sequences simultaneously, utilizing multiple primers/probes working at the same annealing temperature, and temperature-mediated DNA polymerase, in a thermal cycler. Preferably, “padlock probes”, also known as “circularizable oligonucleotide probes” or c-probes, are utilized. Multiplex PCR, in isolation, is generally known; see for example: Nilsson, M., Malmgren, H., Samiotaki, M., Kwiatkowski, M., Chowdhary, B. P., & Landegren, U. (1994). Padlock probes: Circularizing oligonucleotides for localized DNA detection. Science, 265(5181), 2085-2088; Deng J, Shoemaker R, Xie B, Gore A, LeProust E M, et al. (2009) Targeted bisulfite sequencing reveals changes in DNA methylation associated with nuclear reprogramming. Nat Biotechnol 27: 353-360; Akhras M S, Unemo M, Thiyagarajan S, Nyren P, Davis R W, et al. (2007) Connector inversion probe technology: a powerful one-primer multiplex DNA amplification system for numerous scientific applications. PLoS One 2: e915; Krishnakumar S, Zheng J, Wilhelmy J, Faham M, Mindrinos M, et al. (2008) A comprehensive assay for targeted multiplex amplification of human DNA sequences. Proc Natl Acad Sci U S A 105: 9296-9301; Ball M P, Li J B, Gao Y, Lee J H, LeProust E M, et al. (2009) Targeted and genome-scale strategies reveal gene-body methylation signatures in human cells. Nat Biotechnol 27: 361-368; all incorporated herein by reference.

The multiplex step 64 includes annealing arms with “CpG” to target methylated DNA template or “TpG”, where T is a converted cytosine, to target unmethylated DNA template. The assumption is that in either case of using “CpG” or “TpG”, the region of interest is different in a diseased state compared to the normal cell DNA.

In an example embodiment, padlock probes, that is, primers that overlap with the CpG dinucleotide at the 3′ end of the extending primer and at the 5′ end of the ligating arm were selected. In one example, there is at least one converted cytosine in a non-CpG context at the 3′ extending end and 5′ ligating arm of the padlock probe. Of course, a person of skill in the art would realize that padlock probes are not an essential element of the invention; any other known DNA amplification method would likely work to varying levels; more particularly, any known multiplex DNA amplification method would likely work to various levels of effectiveness, especially by utilizing methodology that makes the extending arm and ligating arm resistant to the exonuclease enzymes. For example, adding a thiol group at the 5′ end of the extending probe and a thiol group to the 3′ end of the annealing arm will make these probes resistant to one type of exonuclease, 5′-3′ exo or 3′-5′ exo only. When both probes are ligated there is resistance to both types of exonuclease enzymes. As further described in the specific examples, below, the multiplex PCR step 64 can optionally be replaced by a step of two sequential linear amplification, using tailed primers. Each linear amplification is performed with a pool of uni-directional primers such as reverse primers of all target regions, followed by purification to remove unused primers. This step is followed by another round of linear amplification with a pool of forward primers against the target regions, and thereby, all the target regions have at both ends-5′ and 3′ of the fragment—a set of tail, for which a primer-set (universal primers) can be used to amplify all the target regions of interest simultaneously. Generally, proof reading DNA polymerases, as a class, and for example, DNA polymerase I, perform well for extending a padlock 3′ arm and ligating to the 5′ arm. However, proofreading DNA polymerases may not work effectively with BS converted gDNA, due to the presence of uracil, which is not typically found in gDNA. Certain proof reading polymerases are in fact known to have a “uracil recognition arm” which stalls the polymerase on encountering uracil. (Greagg, M A, M J Fogg, G Panayotou, S J Evans, B A Connolly, and L H Pearl. 1999. “A Read-Ahead Function in Archaeal DNA Polymerases Detects Promutagenic Template-Strand Uracil.” Proceedings of the National Academy of Sciences of the United States of America 96 (16): 9045-50. doi:10.1073/pnas.96.16.9045, incorporated herein by reference).

### Step 5: Verification Step 66

An exponential PCR amplification with universal primers for repeats (Table 6) can be optionally used to verify both a successful multiplex PCR step 64, as well as that there was sufficient DNA in the sample material obtained in step 50 and also, that the sample DNA survived the bisulfite reaction in step 54. In other words, exponential PCR amplification with universal primers for repeats can be optionally used to confirm that there is sufficient DNA obtained from steps 1-4 to proceed with further testing of the sample, for example, for determination of the presence of methylated or hypermethylated gDNA.

### Step 6: Optional Template Improvement Step

In the event of a negative result in the verification step, meaning insufficient DNA to analyze, there is a possibility that not enough template circles are present in the sample. The use of a single universal primer with phi29, a rolling circle polymerase, will improve the template amount for the downstream exponential amplification step. Phi 29 polymerase enzyme is as shown, for example, in Johan Banér, Mats Nilsson, Maritha Mendel-Hartvig, Ulf Landegren; Signal amplification of padlock probes by rolling circle replication, Nucleic Acids Research, Volume 26, Issue 22, 1 Nov. 1998, Pages 5073-5078, https://doi.org/10.1093/nar/26.22.5073, incorporated herein by reference.

By altering the common backbone of a padlock probe, different sets of genomic regions can be investigated in the same reaction. For the given example, we added a set of probes for generating signals from the ALU and LINE repeats and another set for targeting unique regions (Table 5).

The repeats represent approximately 70% of the human genome. The probes against repeats will serve as an endogenous control to verify whether sufficient DNA is present after bisulfite treatment and that all the steps were performed correctly. These repeats can further be analyzed in a separate Next-generation Sequence run, or the multiplex PCR step 64 can be repeated after the template improvement step 68.

### Step 7: Genomic Analysis 70

The amplified gDNA can be subjected to genomic mapping analysis 70, with the presence of amplified gDNA indicative of a presence of hypermethylated DNA regions in the original extracted sample. The amplified gDNA can be sequenced, for example, to determine which hypermethylated DNA regions were present in the original extracted sample, in order to design personalized medical therapy based on the specific tumor suppressor gene or set of genes that are hypermethylated.

Thus, the present method offers improvements to prior art multiplex PCR analysis, due primarily to the addition of linear amplification step 58, which converts the uracil, and allowed for most of the proof reading polymerases to function very well, without influencing the specificity to capture the region of interest. The resultant amplified DNA is extremely and selectively rich in amplification of methylated or unmethylated DNA templates of a differential region-alias a biomarker, and can be utilized to determine whether such DNA exists in a sample, which may be used as a predictor of disease, such as cancer.

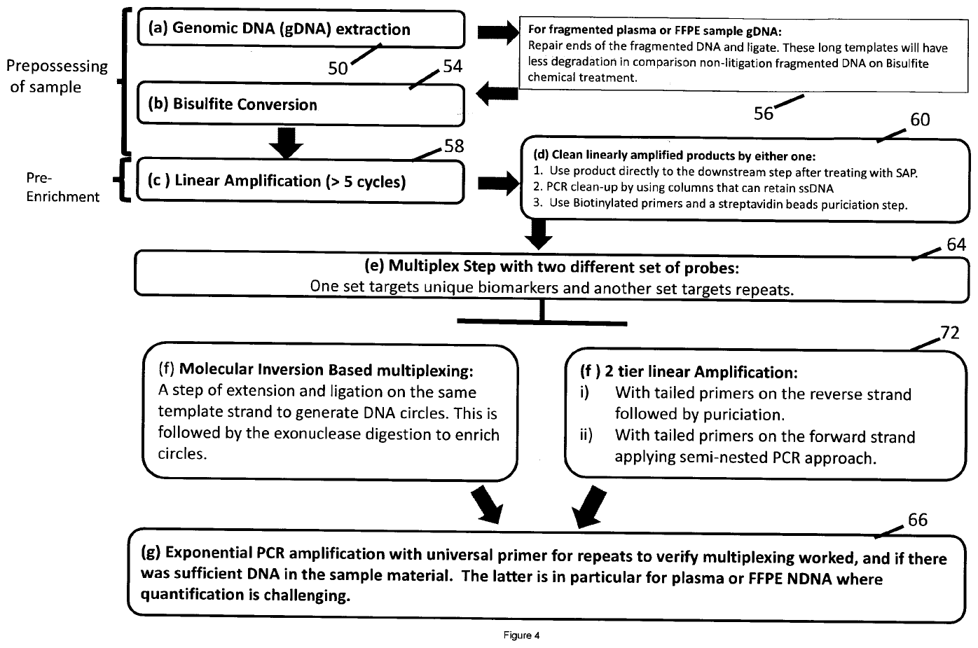

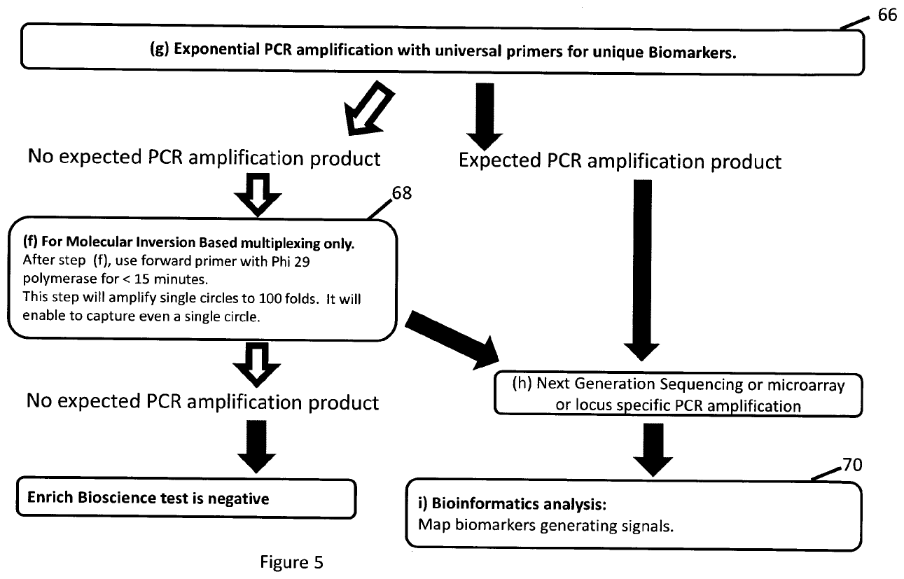

FIGS. 4 and 5 show an alternative, more detailed representation of FIG. 2.

### EXAMPLE 1

### Optimization of Example Method

Example Experimental Aims: (a) To optimize a number of cycles required for linear amplification in order to obtain robust amplification from the downstream multiplex; (b) Analyze influence of annealing time of probes on multiplex amplification for the traditional multiplex approach compared to the modified approach; (c) Analyze the detection limit of the traditional multiplex approach and the modified approach; (d) Analyze lower limit for number of regions in multiplex method and usage of phi29 polymerase in the event when low number of regions are generating signals; (e) Analyze cancer cases—an affected tumor, healthy adjacent and plasma samples and later, analyzing by end-point PCR; (f) Usage of 3 step linear amplification as an alternative for multiplex-padlock.

### Example Aim A

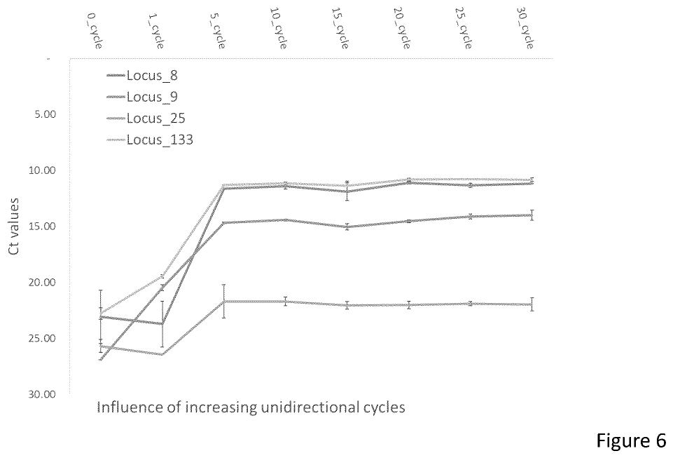

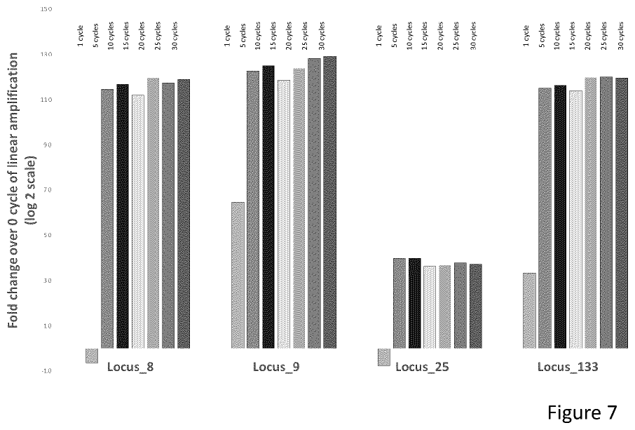

With reference to FIGS. 6 and 7, linear amplification was performed with different cycle numbers ranging from 1 to 30 cycles. A control of 0 cycles, where no linear amplification was performed, was used as a negative control, which in the entire scenario showed negligible amplification in the Example method (data not shown). Each scenario was performed in duplicate and the mean values were plotted, see FIG. 6. The exponential PCR product obtained after 8 cycles of amplification with a universal primer pair, were later subjected to the locus-specific PCR without diluting, a variation of the Enrich Example protocol described below in Examples 2 and 3.

As disclosed in FIGS. 6 and 7, four locus-specific real-time assays were performed to determine the robustness of the enrichment, as locus 8, locus 9, locus 25 and locus 133.

Example Conclusion 1: With reference to FIGS. 6 and 7, all 4 loci (locus 8, locus 9, locus 25 and locus 133) showed that amplification from 5 cycles and onwards had a similar amplification, (i.e. a plateau), suggesting that there is a negligible influence of cycle number to the amplification efficiency of the example disclosed Enrich method.

### Example Aim B

Aim B was to test the influence of incubation time, at the annealing temperature, on PCR amplification efficiency. For simplicity, we used an example linear amplification of 10 cycles for the example disclosed Enrich method.

Different annealing incubation protocols were tested for the traditional method and for the example disclosed Enrich method. Table 1 below describes 3 different incubation protocols (Protocol_01, Protocol_02, Protocol_03):

TABLE 1
Time in hrs

| Temperature | | | |
|---|---|---|---|
| (° C.) | Protocol_01 | Protocol_02 | Protocol_03 |
| 95 | 0.05 | 0.05 | 0.05 |
| 90 | 0.75 | 0 | 0 |
| 85 | 0.75 | 0 | 0 |
| 80 | 0.75 | 0.25 | 0.08 |
| 75 | 0.75 | 0.25 | 0.08 |
| 70 | 0.75 | 0.25 | 0.08 |
| 65 | 0.75 | 0.25 | 0.08 |
| 60 | 0.75 | 0.25 | 0.08 |
| 56 | 3 | 1 | 0.5 |
| Total (hrs) | 8.3 | 2.3 | 1.0 |

All of the downstream steps were the same for all the 3 protocols for the traditional (prior art) method, as taught by Diep (Diep D, Plongthongkum N, Gore A, Fung H L, Shoemaker R, et al. (2012) Library-free methylation sequencing with bisulfite padlock probes. Nat Methods 9: 270-27, incorporated herein by reference) with minor modifications as described further below, and for the example disclosed Enrich method and both methods were compared on average of 2 technical replicates.

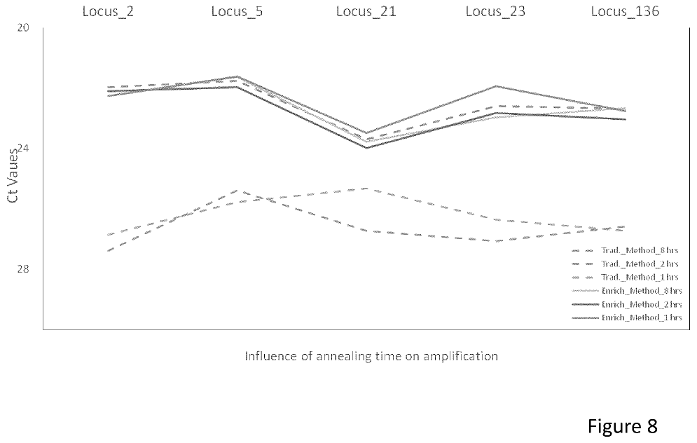

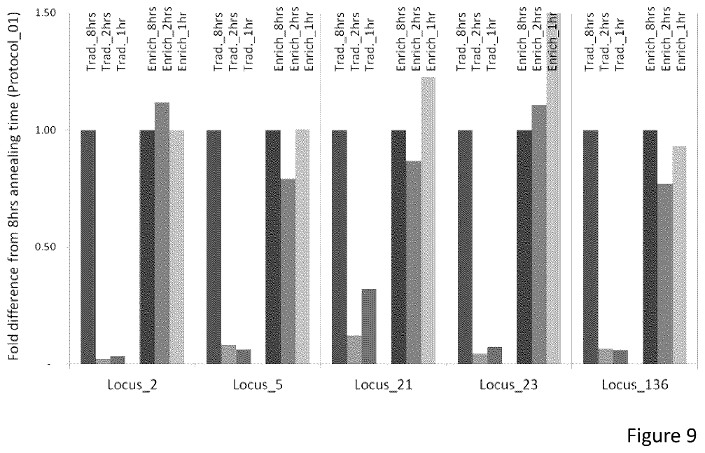

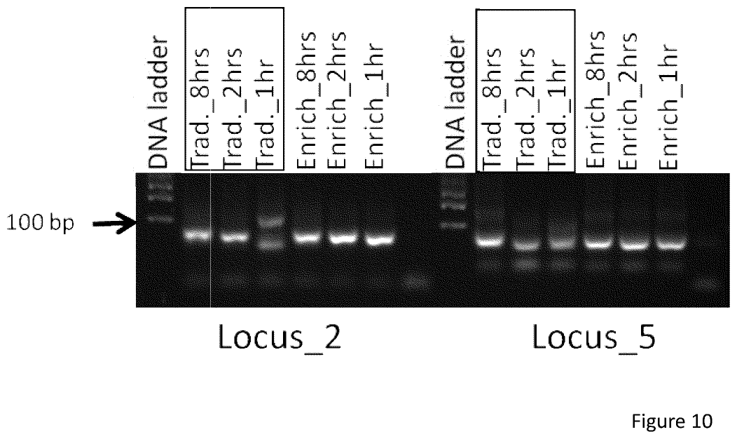

Example Conclusion 2: With reference to FIGS. 8, 9 and 10, the example disclosed Enrich method showed that lowering of the incubation time at the annealing stage has no impact on the downstream amplification step. However, the traditional (prior art) method, lacking the linear amplification step, had significant influence with a reduced incubation time of 2 hrs or 1 hr. Further, not only was the amplification low, but also non-specific amplification was observed at a reduced annealing time (locus 2, the 1 hr of incubation for the annealing step of the traditional method). The traditional (prior art) method follows the protocol described in Diep et al, with a modification that extension and annealing arms contain CpG to target methylated templates. The annealing and extension arm sequences for both the methods, traditional and Enrich, are complementary and target the same bisulfite converted template.

Aim C: To estimate the detection limit of the two methods (the disclosed example Enrich method compared to the traditional (prior art) method).

Similar to Aim B, we used an example linear amplification of 10 cycles for the example disclosed Enrich method.

Methylated genomic DNA and unmethylated DNA (commercially available) were bisulfite converted respectively. The BS converted DNA was measured on Nanodrop (3 times) to have a robust estimate. Thereafter, methylated DNA was diluted to attain 100 ng, 10 ng, 0.025 ng, and 0.00125 ng of DNA. Thereafter, a serial dilution (1:20) was performed 4 times, in order to attain the last dilution to be 7.8 ×10\hat{ }−9 ng of methylated DNA. These dilutions translate the proportion of methylated DNA (or cancer DNA) as shown in Table 2, below:

TABLE 2
Serial Dilution Table

| | | Number of | Proportion of |
|---|---|---|---|
| | Dilutions | methylated | methylated |
| | (ng of DNA) | genome | genome |
| 1 | 1.00E+02 | 1.67E+05 | 1.67E+05 |
| 2 | 1.00E+01 | 1.67E+03 | 1.67E+03 |
| 3 | 2.50E−02 | 4.17E+00 | 4.00E+00 |
| 4 | 1.25E−03 | 2.08E−02 | 1 in 48 |
| 5 | 6.25E−05 | 1.04E−03 | 1 in 960 |
| 6 | 3.13E−06 | 5.21E−05 | 1 in 19200 |
| 7 | 1.56E−07 | 2.60E−06 | 1 in 384000 |
| 8 | 7.81E−09 | 1.30E−07 | 1 in 7680000 |

Note:
1 cell has 6.6E−03 ng of genomic DNA

The diluted DNA sample was converted to the estimated number of diseased cells (which is methylated at specific regions) in a pool of normal cells. A single cell has 6 picograms of DNA, which was used to calculate the fraction of methylated DNA that is present in a pool of DNA from the normal cells.

BS converted unmethylated DNA was supplemented to get a final concentration of 100 ng. Each dilution was then subjected to traditional (Trad.) multiplex method or to the presently disclosed method (Enrich).

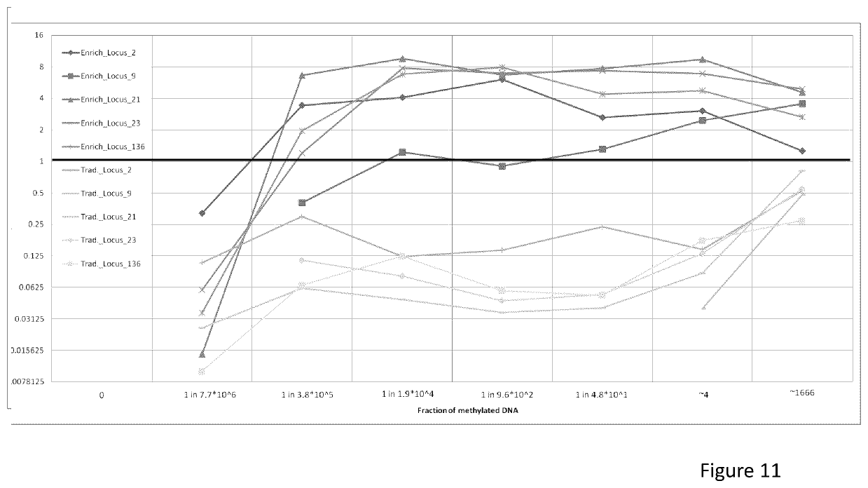

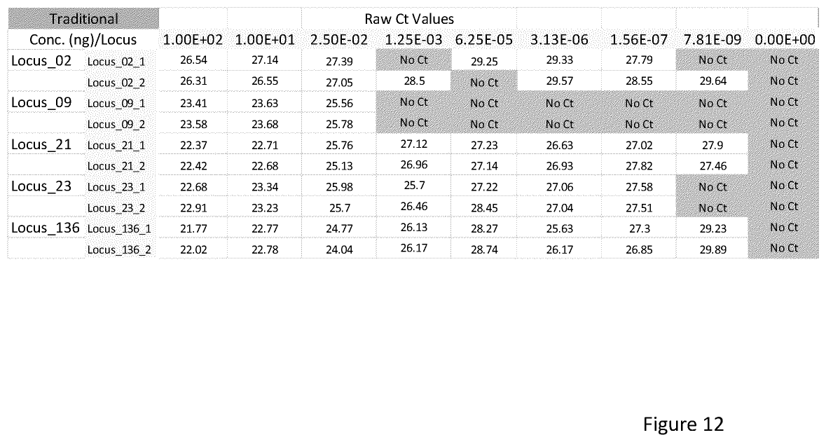

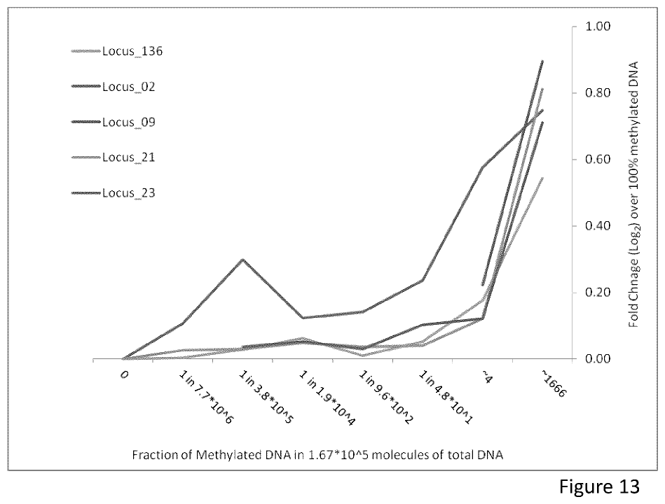

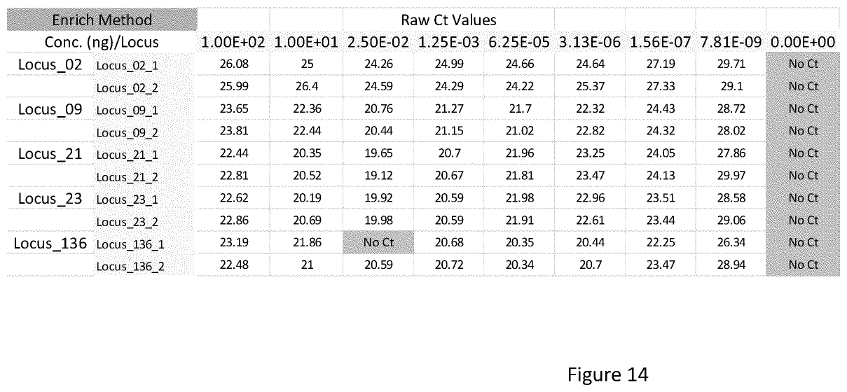

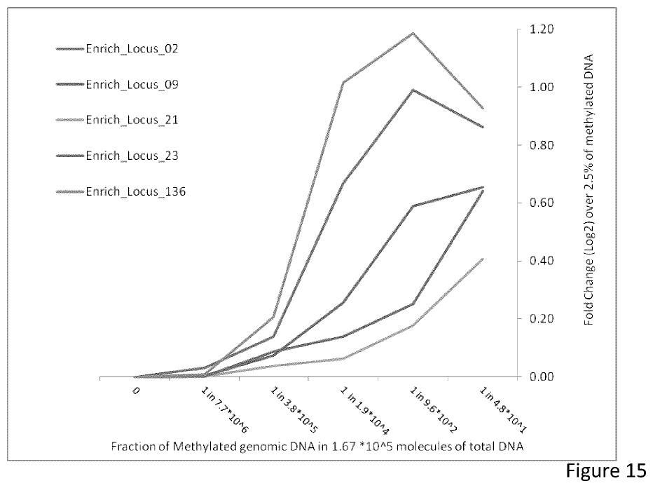

Fold change of each sample was calculated against 100% methylated DNA sample (2\hat{ }−(Ct of a sample−Ct of 100% methylated sample) or 2.5% methylated DNA sample for the example Enrich method, and plotted as a line plot (See FIGS. 11-15). For the example traditional method, 100% methylated DNA was used. However, for the example Enrich method 2.5% methylated DNA was used, as that performed better than alternative dilutions such as 100% and 10%.

Example Conclusion 3: With reference to FIGS. 11-15, a robust amplification was observed for the example disclosed Enrich method for all the dilution from 1 in 3.8 E10\hat{ }5 and above, and similar, but weak signals, were also observed for the traditional method. Further dilution of 1 in 7.7E10\hat{ }6 showed random amplification of the loci in both the traditional method and example disclosed Enrich method.

These results suggest that working with a low amount of DNA is challenging, however, the example disclosed Enrich method is efficient in capturing the signals across different loci, while the traditional method may fail to generate signals from all the expected loci. The detection limit in both the methods is consistent with the cited literature i.e 1 in 10\hat{ }5 (Volik, Stas, Miguel Alcaide, Ryan D Morin, and Colin C. Collins. 2016. “Cell-Free DNA (cfDNA): Clinical Significance and Utility in Cancer Shaped by Emerging Technologies.” Molecular Cancer Research: MCR 14 (778): molcanres.0044.2016. doi:10.1158/1541-7786.MCR-16-0044, incorporated herein by reference). Of note, to gain information on cancer (or any other disease) signature, different loci are required to have robust amplification, which was only observed in the Example disclosed method.

Example: Aim D Analysis of a minimum number of regions in the multiplex method and usage of the phi29 polymerase to amplify signals from a low number of regions contributing as a template.

Similar to Aim B, we used linear amplification of 25 cycles for the example disclosed in the Enrich method, utilizing the template improvement step 68.

The number of primers used for linear amplification was 0, 5, 10, 15, 20 and all 155 on a 10 ng of control bisulfite treated DNA.

Exponential amplification with universal primer set was performed before and after phi 29 polymerase reaction.

One half of the processed sample material was subjected for phi29 polymerase reaction in the presence of 1× Cutsmart buffer (NEB), 10 μmoles of dNTPs, 1 μmoles of forward universal primer in a reaction volume of 30 ul. The reaction was heat denatured at 98° C. for 2 minutes before adding 0.2 U of phi29 polymerase (NEB) and incubated at 30° C. for 15 minutes followed by heat inactivation at 80° C. for 20 minutes.

The forward primer was not exo-resistant, as suggested elsewhere: NEB protocol for phi 29 polymerase.

Example Conclusion 4: The padlock-multiplex method has a limitation for a number of regions that contribute to generating signals visible on a size separation agarose gel, however, the locus-specific end-point PCR showed that regions were present and survived all the steps of the method of the invention presented here. With an additional step of phi29 polymerase, the multiplex PCR band was visible at the correct size. The phi29 polymerase step is an optional step and is only recommended when no PCR product is visible on an agarose size separation gel at the first instance.

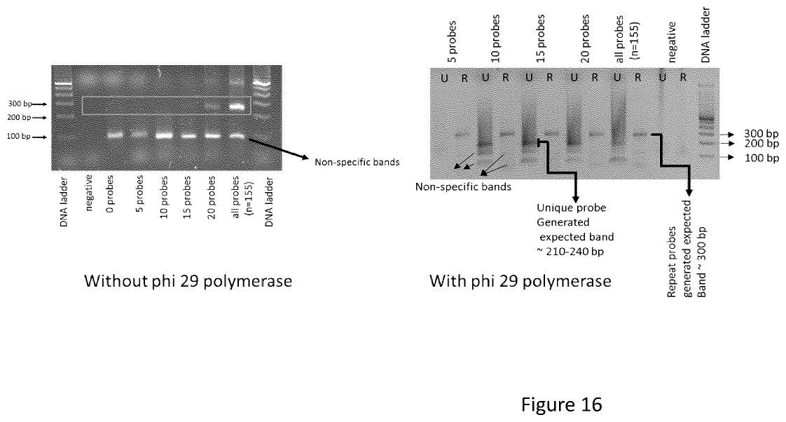

Results are shown in FIG. 16, which shows the PCR products from two sets—unique regions (U) and repeat probes (R) when a different number of padlock probes were used (0-155) with and without the phi29 polymerase step (the template improvement step 68). In this particular instance, when the template improvement step 68 was used, the PCR band was visible, at the correct size for padlock probes lower than 15. The use of the template improvement step 68 was also shown to generate higher sized amplicons, resulting in a larger smear. It is a known artifact of rolling circle phi29 polymerase. Nelson, J. R. (2013). Random-Primed, Phi29 DNA polymerase-based whole genome amplification. Current Protocols in Molecular Biology, (SUPPL.105). ttp://doi.org/10.1002/0471142727.mb1513s105, incorporated herein by reference.

Alternately, Klenow fragment polymerase can be used instead of a phi29 polymerase. The reaction conditions are same as described for phi29 polymerase reaction, except that the incubation temperature is at 37° C. for 15 minutes followed by heat inactivation at 80° C. for 20 minutes.

Example: Aim E Analyze cancer cases—an affected tumor, healthy adjacent and plasma samples and later, analysis by end-point PCR.

Thirty colorectal-cancer matched cases, for sex, age and stage of cancer, were obtained from the two biobank—Alberta Biobank (n=14) and Ontario Tumor Biobank (n=16). A case is defined as affected tumor tissue, adjacent healthy tissue and plasma sample from the same individual. Samples were obtained after ethics approval (IRB Tracking Number:16181-09:34:1216-07-2018).

Genomic DNA was extracted using a genomic DNA isolation kit from Qiagen and quantified using nanodrop for 260/280 and 260/230 ratios. The quality of tissue DNA was also assessed on an agarose size separating gel.

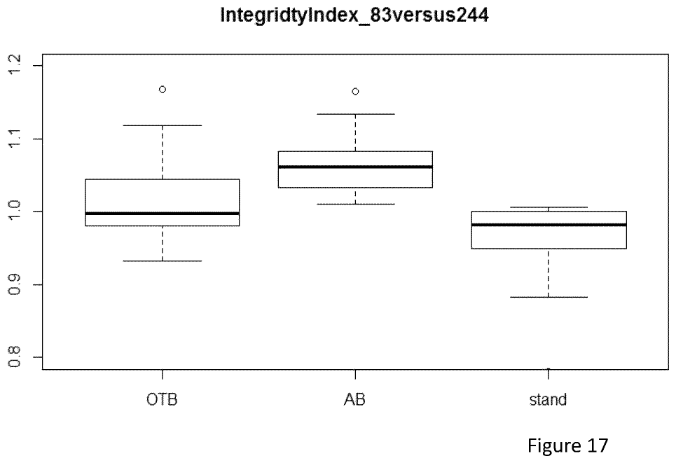

DNA was assessed for fragment size by using 83 bp and 244 bp of ALU repeats real-time PCR as described in Bedin et al 2017 (Bedin, Chiara, Maria Vittoria Enzo, Paola Del Bianco, Salvatore Pucciarelli, Donato Nitti, and Marco Agostini. 2017. “Diagnostic and Prognostic Role of Cell-Free DNA Testing for Colorectal Cancer Patients.”International Journal of Cancer 140 (8). Wiley-Liss Inc.: 1888-98. doi:10.1002/ijc.30565, incorporated herein by reference). The results were shown in FIG. 17, with the Ontario Tumor Biobank (OTB) samples compared to the Alberta Biobank (AB) samples and a standard control (stand).

All samples DNA were randomized before subjecting to the Enrich method.

Two sets of multiplex padlock probes were used: One set target methylation specific unique biomarkers implicated in different cancer types, while the second set target was to target the ALU and LINE repeats. These both sets had different primer pairs for exponential amplification (Table 6). The probes for repeats was an endogenous control.

After step 62, ⅙^{th} of the processed samples were amplified with universal primers (step 64) using KAPA SYBR fast Universal qPCR Master Mix (Kappa Biosystems) under the following conditions:, 98° C. for 2 m, 95° C. for 10 s, 22 cycles of 95° C. for 10 s; 60° C. for 10 s, and 72° C. for 20 s, followed by the step of 72° C. for 2 minutes followed by the melt curve analysis.

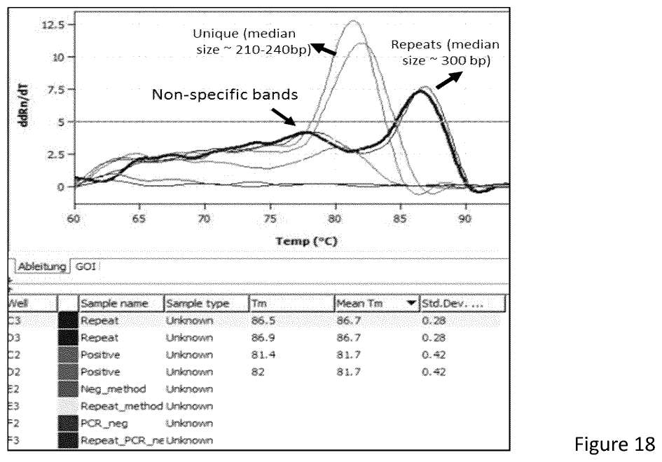

The melt curve analysis of the unique regions and repeats is shown in FIG. 18, showing discrete PCR product size of unique regions (ranging from 210-240 bp) and of the repeats product size (approximately of 300 bp).

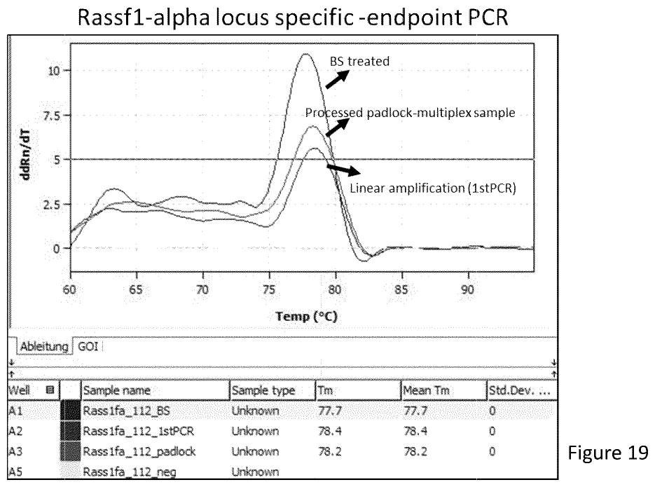

The multiplexed product was diluted to a hundred folds and a locus specific end-point PCR was performed. A melt curve analysis shown in FIG. 19 is an example demonstrating the specificity of the amplicon. The PCR amplification of RASS1Fa from different —after bisulfite treatment (step 54), after linear amplification (step 58) and purification, and after the multiplexed amplification (step 64).

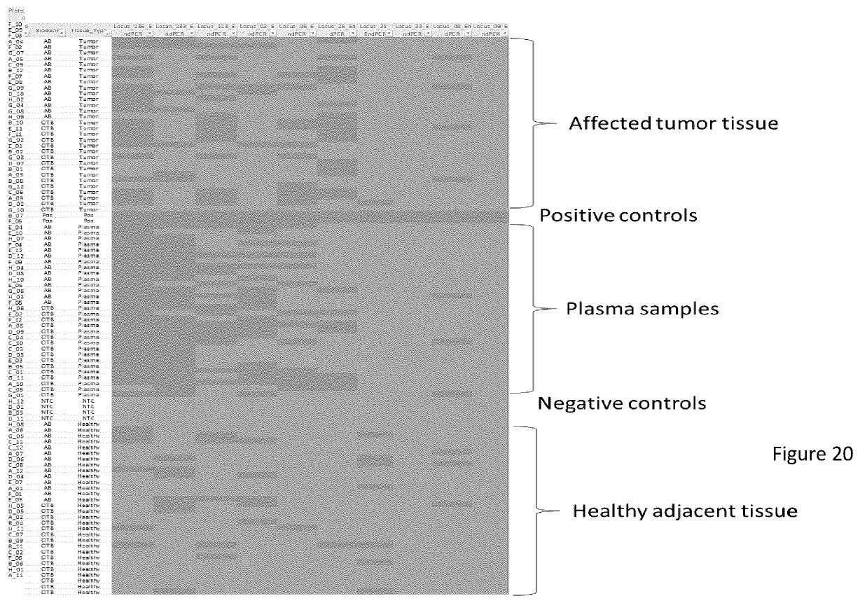

Example Conclusion 5: In all scenario, the generated PCR products have a close melting temperature (Tm) suggesting similar sized and nucleotide composition of the PCR product. We tested for the end-point PCR amplification and meltcurve analysis on ten different loci across all cancer case samples shown in FIG. 20.

Each row of FIG. 20 represents a sample, while each column is a biomarker. The biomarkers used were on the gene promoters of CDKN2a, SEPT9, ARF1, BRCA1 and RASSF1a. The darker color (orange color) indicate when a region was amplified, as seen from the amplification plot and melt curve analysis and confirmed on an agarose gel size separation. The lighter color (grey) indicate when no amplification was found, or a wrong sized PCR product was present. As can be seen from the Figure, many of the affected and blood plasma sample DNA showed region specific amplification. Interestingly, the plasma samples showed biomarkers more often than the affected tissue, while healthy tissues had amplification on sporadic samples. This observation may relate to the fact that a tumour is heterogeneous and from tissue, only a small tissue section is used for the analysis. While the blood samples reflect that an entire tumour sheds its DNA into the blood; and these preliminary results suggest that blood sampling, using the Enrich method, may be a better indicator of whether a tumour is present.

Example Conclusion 6: The method demonstrated that biomarkers reported with colon cancer had robust signals from various samples. Without the phi29 step, except positive controls, none of the samples showed the presence of the expected band on a size separating agarose gels. With phi29 step, many samples—plasma, healthy and affected tissue DNA samples showed expected sized PCR products. From the locus-specific end time PCR and melt curve analysis, multiple plasma samples had amplification of an associated locus with colon cancer compared to the affected tissue, while healthy adjacent tissue samples had PCR amplification from the sporadic samples only (FIG. 20).

Example: Aim F Usage of three-step linear amplification as an alternative for multiplex-padlock.

In this methodology, the multiplex PCR step 64 was replaced with two further linear amplification steps (see FIG. 4, optional step 72). In FIG. 4, this is referred as a “2 tier linear amplification”, though, when you include step 58, there is in total three linear amplification steps.

Similar to Aim B, we used a linear amplification of 25 cycles for the example disclosed Enrich method.

Linearly amplified products purified, and subjected to converting single-stranded DNA from the first step of linear amplification to the double-stranded DNA.

The forward-tailed unidirectional primers pool 20-80 attomole of each primer (design of primers as described below) in 20 μl of Cutsmart buffer (NEB) containing 2 micromolar of dNTPs from Thermo Sci. The reaction was heat denatured at 98° C. for 2 minutes and later, supplemented with 0.5 U of Klenow fragment polymerase (NEB) enzyme and incubated at 37° C. for 30 minutes followed by heat inactivation at 80° C. for 20 minutes.

The samples were treated with Exonuclease I enzymatic reaction to degrade all the leftover primers as well as any single stranded-template. 8 U of exonuclease I (USB), and 0.05 U of Uracil-Specific Excision Reagent (USER; NEB)) in exonuclease buffer III (USB) were incubated at 37° C. for 120 minutes followed by inactivation at 80° C. for 20 minutes and 95° C. for 5 minutes.

Samples were column purified Qiagen using PN buffer and eluted in 10 ul EB buffer (Qiagen).

Samples were then subject to the reverse-tailed unidirectional primers. 20-80 attomole of each primer (design of primers as described below) in 20 μl of Thermo Sci. buffer (HF 5×) containing 2micromolar of dNTPs, and Phusion proofreading polymerase (Thermo Fischer) under the following conditions: 98° C. for 2 minutes; 5 cycles of 95° C. for 10 s, 60° C. for 10 s, and 72° C. for 20 s; followed by 72° C. for 2 minutes.

5 μl of processed samples were amplified using a universal primer set as stated elsewhere.

Example Conclusion 7: Expected band size was observed from the two-tiered multiplexing method on a size separation agarose gel. A few locus-specific amplification were tested for confirming the target amplification of the desired regions.

The following is an example protocol for the example method disclosed herein.

### EXAMPLE 2

### Example Protocol for Preferential Amplification

### Step 1: Genomic DNA Isolation

In one example, Genomic DNA was obtained and purified from a blood sample, using a QIAamp Circulating Nucleic Acid Kit (Qiagen NV, The Netherlands).

In one example, Genomic DNA was obtained and purified from a tissue sample, using a QIAamp DNA Kit (Qiagen NV, The Netherlands).

In one example, the human methylated and native blood DNA (Human blood DNA was obtained from 2 sources (Thermo Sc. and Roche respectively). The native blood DNA was then subjected for whole genome amplification (phi29 polymerase and exo-resistant random primers; Thermo Sc.) to erase all the DNA methylation and to obtain an unmethylated control DNA.

A high quality of genomic DNA, A260/A280 ratio of greater than 1.8 and A260/230 ratio of greater than 2.0, is recommended for DNA methylation analysis.

### Step 2: Bisulfite Conversion Step 54

500 ng, methylated and unmethylated, of gDNA, was bisulfite converted (BS) with EZ DNA Methylation-Gold™ Kit (Zymo research) according to the manufacturer's protocol. BS converted gDNA was quantified on the Nanodrop with the option of single stranded DNA (Thermo Sc.) and 100 ng of BS converted DNA was used for the downstream steps.

### Step 3: Linear/Unidirectional PCR Amplification Step 58

1-100 ng of BS converted DNA was annealed to the unidirectional primers pool 20-80 attomole of each primer (design of primers is described below) in 10 μl of Universal PCR Master Mix containing 2 micromolar of dNTPs from Thermo Sci. using modified proof reading polymerase that can tolerate uracil in the template (Phusion U from Thermo Fischer or any other non-proof reading polymerase can be used) under the following conditions: 98° C. for 2 minutes; 1-30 cycles of 95° C. for 5 s, 55° C. for 10s, and 72° C. for 20 s; followed by 72° C. for 2 minutes. Example primers are shown in Table 3.

TABLE 3
A few primers used for linear
amplification of the example assay

| | |
|---|---|
| ARF1_SEMI_2 | AAACACCCTACCCCGA |
| BRCA1_SEMI_8 | TTTCCGTTACCACGAA |
| BRCA1_SEMI_9 | TCCCCCACTCTTTCCG |
| CDKN2A_SEMI_20 | CTTCCCACCCTCAACG |
| CDKN2A_SEMI_21 | CATTCGCTAAATACTCGA |
| CDKN2A_SEMI_22 | GACTCTAAACCCTACGC |
| RASSF1_SEMI_113 | CCAAACAAACGAACGCG |
| SEPT9_SEMI_133 | CTACAAAAAAACCCTACG |
| SEPT9_SEMI_136 | CCTTCCCCGAACGC |

Linear amplified products were directly used for the downstream application after treating with shrimp alkaline phosphatase (SAP, Thermo Fisher), which degrades any unused dNTPs in the reaction. 1 U of SAP is added directly to the PCR mix, incubated at 37° C. for 20 minutes, and later, the SAP was inactivated at 80° C. for 10 minutes.

Alternatively, the PCR products can go a cleanup step using PCR purification columns (Zymo Research).

Unidirectional primers can also be tagged with 5′-biotin for a cleanup procedure using standard biotin—streptavidin protocols.

At this example stage, complimentary strands are ready for padlock processing.

### Step 3B: Optional/Modified Protocol to Capture Genomic Regions with Padlock Probes (Optional Genomic Region Capture Step 62)

11 μl of unidirectional PCR product was annealed to the padlock probe pool (20-80 attomole of each probe) in 1× Ampligase buffer (Epicentre) in a total volume of 10-20 μl with the following incubation conditions: 95° C. for 3 minutes, 85° C. for 30 minutes, and 5° C. lowering of temperature till the 56° C., and incubation at 56° C. for 120 minutes.(this step can be held also for overnight annealing).

It was found that, preferably, each probe library should be optimized for BS-DNA to probe ratio (1 ng to 0.05 ng of probes per 50-200 ng of BS-DNA).

The annealed DNA template was subjected to extension and ligation in the presence of 1× Ampligase® buffer (Epicentre), 10 pmoles of dNTPs, 20 μmoles of NAD+, 1 U of DNA polymerase (proof reading polymerase-modified or no modification for the Uracil recognition arm) and 2.5 U Ampligase (Epicentre), in a reaction volume of 25 μl. Extension and ligation were performed at 56° C. for 60 minutes, followed by 72° C. for 20 minutes (the step at 56 degree can be held for longer incubation period).

Although any suitable proof-reading polymerase can be used for this multiplex step, we found that Phusion® and Phusion® U polymerase (Thermo Sc.) worked very well.

In one example we also found that proof reading polymerases, such as Phusion® polymerase will work for the padlock step, in the traditional method, on the Bisulfite converted template in contrast to the literature which suggests that proof reading polymerases stall at the uracil nucleotides.

Alternatively to Ampligase®, other ligases, such as 9° N DNA ligase (NEB), also function well in the protocol.

The ligated products formed single stranded circles and are resistant to exonucleases. 5 μl cocktail of exonucleases was used to degrade the non-circularized DNA and the unused probes; 8 U of exonuclease I (USB), 40 U of exonuclease III (USB), 6 U of RecJf (NEB), 0.05 U of Uracil-Specific Excision Reagent (USER; NEB) and 2.5 U of lambda exonuclease (NEB) in exonuclease buffer III (USB) were used to enrich circularized templates at 37° C. for 120 minutes followed by inactivation at 80° C. for 20 minutes and 95° C. for 5 minutes.

In one example we noted that use of exonuclease enzymes in the cocktail generally resulted in a higher efficiency of linear DNA digestion. The inclusion of USER, an enzyme that cleaves uracil residues present in the template BS DNA provided better results for the downstream amplification.

Step 4: PCR Amplification and Validation of the Integrity of the Generated Amplicons Generation of Illumina® Library for Sequencing (Multiplex PCR step 64 and Verification Step 66)

7.5 μl of the exonuclease digest was amplified in KAPA SYBR fast Universal qPCR Master Mix with 7.5 μmoles of each Universal—forward and—reverse primers (bar coded) (see Table 6) in a volume of 15 μl. The PCR amplification was performed in triplicate as follows: initial denaturation of 98° C. for 2 minutes, followed by 95° C. for 5 s, 60° C. for 10 s, 72° C. for 10 s for 8-22 cycles and with a final extension at 72° C. for 10 minutes.

This step was optionally coupled with real-time PCR to monitor the amplification with the number of cycles required.

The obtained PCR products were then diluted in water (1:100) and 1 μl of the diluted 1st cycle product was used as a template to perform locus-specific amplification in KAPA SYBR fast Universal qPCR Master Mix and with 7.5 μmoles of primer-pair in a volume of 10 μl. The PCR amplification was done as follows: initial denaturation of 98° C. for 2 minutes, followed by 95° C. for 5 s, 60° C. for 10 s, 72° C. for 10 s for 30 cycles. A melt curve was included at the end of real-time PCR to analyze the accuracy of the product generated. It was also verified by size separation agarose gel electrophoresis.

### Primer Design for Unidirectional or Padlock PCR

Any number of probes can be designed using the following example considerations:
Genomic DNA is masked for the repeat, common-SNP and segmental duplicates.
Sequences were theoretically bisulfite converted i.e. all C converts to U except at the CG dinucleotide sequence.
18-20 mer primer was designed from publically available primer designing tools. Primers with at least one “CpG” within the last 5-8 nucleotides from the 3′ end were selected to capture methylated target region, while for targeting non-methylated region, the converted “TpG” is selected. Strategies can also develop to capture both methylated and unmethylated templates of the locus by excluding “CpG” or “TpG” in the primer, as mentioned for standard designing of primers for a BS converted genome.
Primers were selected for containing preferably at least 1 “CpG” within the primer and no more than 5 “CpG” in the primer sequence.
Primers were selected for preferably an annealing temperature of 40-60° C.
Stretches of any nucleotide more than 5 in a row were generally avoided, especially towards the 3′ end.
At least 2 converted C to U were preferably included in the primer.

The designed probes were synthesized from a nucleotide synthesizing company such as Operon, Oligodt, or Thermo Sc. etc. Probes may be of 18-150 bp in length, preferably around 100 base pairs. Further, any number of probes can be used; we have tested 5 to 40,000.

For multiplex oligonucleotide-design, the bisulfite converted template and with unidirectional step was transformed to the complementary strand. The primers were then designed on this newly formed complementary strand using following criteria:
In one example, PCR product length was in the range of 80-120 bp, excluding primer pair length, and this will yield a PCR product of 120-160 bp after multiplexed method. This criterion was adopted with the consideration that plasma DNA or cell free circulating DNA has a length of 100-500 bp.
18-20 mer primer was designed from the publically available primer designing tools (primer 3, Oligo). Primers were then selected that have at least one “CpG” within the last 5-8 nucleotides from the 3′ end to target methylated regions, while a converted “TpG” was adopted at the same position to target unmethylated DNA.
In one example, at least one “CpG” was recommended at the 5′ end of the ligation arm, however, no more than 5 CpG in the primer was recommended.
In one example, theoretical annealing temperature of the primer pair was selected from about 58-65° C. and the annealing temperature difference between the primers pair were selected to be within 20° C. of one another.
In one example, stretches of any nucleotide more than 5 in a row were avoided, especially towards the 3′ end of the extending arm or 5′ end of the ligation arm.
The primer pairs following above criteria are selected and a random 4-7 N's was included within the common linker to trace back PCR duplicates.
The extended region that has no CpG was excluded from the primer list.

The designed probes were synthesized from an oligo-service (LC Sciences) company that provides cost effective oligos. If ordered from LC

Sciences then probes require additional processing step (stated below) and these probes have additional linkers at the 3′ and 5′ end.

However, lower number of primers can be ordered from any nucleotide synthesizing company such as Operon, IDT or Thermo Sc. etc.

Note: In mammals, CpG dinucleotide is the preferred sites for the DNA modifications.

### Preprocessing of Padlock Probes

In one example, prior to their use, padlock probes were processed as below:

2 nM of mixed template oligonucleotides was PCR amplified in the presence of 100 nM each of Adopter Forward primer and Adopter reverse primer (Table 4: set of A or B), and 10 μl of KAPA SYBR fast Universal qPCR Master Mix (Kappa Biosystems) under the following conditions:, 98° C. for 2 m, 95° C. for 5s, 12 cycles of 95° C. for 5 s; 50° C. for 1 minutes; and 60° C. for 30 s, and 72° C. for 10 minutes.

TABLE 4
Primers to generate Padlock probes
Primers to generate
Traditional Padlock probes (Set A)

| | |
|---|---|
| Org_Pri_For | TGCCTAGGGTCTCGACTGGU |
| Org_Pri_Rev | GAGCTTCGGTGCACGCAATG |
| Probe_For | ACCAGTCGAGACCCTAGGCA |

Primers to generate
modified (Enrich) Padlock probes (Set B)

| | |
|---|---|
| revOr_Pri_F | TGCCTAGGCTGAGCAGTGCU |
| revOr_Pri_R | GAGCTTCGCACGTGGCAATG |
| Probe_Rev | AGCACTGCTCAGCCTAGGCA |

The resultant amplicons were purified with Qiaquick PCR purification columns (Qiagen) using PB buffer.

Probes were re-amplified by PCR in 100 reactions (50 μl each) with 0.1 nM of first round amplicons, 100 nM each of Adopter Forward primer and Adopter reverse primer (respective A or B set), and 50 μl of KAPA SYBR fast Universal low ROX qPCR Master Mix (Kappa Biosystems) under the following conditions: 98° C. for 2 minutes, 95° C. for 5 s, 12 cycles of 95° C. for 5 s; 60° C. for 30 s; and 72° C. for 30 s, and 72° C. for 10 minutes.

The resulting amplicons were purified by Qiaquick PCR purification columns (Qiagen) using PB buffer.

The purified PCR amplicons (4 μg) were digested with 10 U of wild type BsrD1 (10 U/μl, NEB) at 65° C. for 1 hour (inactivation at 80° C. for 20 minutes) and followed by lambda exonuclease digestion 2 U per reaction at 37° C. for 1 hour (inactivation at 80° C. for 20 minutes). The digested products were subjected for USER digestion to digest the U present at the 3′ end of the forward primer.

The single stranded DNA was purified using the Qiaquick PCR purification column (Qiagen) using PB buffer. The eluted single strand was hybridized with the oilgo and then digested with Bst1 (NEB) (similarly for the other probe type BsrI (NEB at 65° C.) restriction endonuclease was used) at 55° C. for 20 minutes and the reaction was stopped by adding urea loading dye (Sigma).

The probe molecule sizes ˜70 mer were purified by size selecting on 6% denaturing Urea-PAGE gel (Thermo Fisher) and electro-eluted using D-Tube Dialyzer 6-8 KDa tubes (Millipore).

In one example, single stranded probes were ordered from ThermoFisher and later, the 5′ end was phosphorylated using T4 Polynucleotide Kinase (PNK; Thermofisher) according to the manufacturer's protocol. The 5′ phosphorylation is required for the ligation during the extension and ligation step of the padlock.

In one example, double stranded probes were used in the multiplex step as described in Shen P et al, 2013. (Shen P, Wang W, Chi A-K, Fan Y, Davis RW, Scharfe C. Multiplex target capture with double-stranded DNA probes. Genome Medicine. 2013;5(5):50. doi:10.1186/gm454).

In one example, two tier linear extension and amplification was used before amplification of target regions with universal primer set.

The embodiments of the present disclosure described above are intended to be examples only. The present disclosure may be embodied in other specific forms. Alterations, modifications and variations to the disclosure may be made without departing from the intended scope of the present disclosure. While the systems, devices and processes disclosed and shown herein may comprise a specific number of elements/components, the systems, devices and assemblies could be modified to include additional or fewer of such elements/components. For example, while any of the elements/components disclosed may be referenced as being singular, the embodiments disclosed herein could be modified to include a plurality of such elements/components. Selected features from one or more of the above-described embodiments may be combined to create alternative embodiments not explicitly described. All values and sub-ranges within disclosed ranges are also disclosed. The subject matter described herein intends to cover and embrace all suitable changes in technology. All references mentioned are hereby incorporated by reference in their entirety.

TABLE 5
Common backbone used for different sets of padlock probes.

| | |
|---|---|
| Unique regions probes—targeting | a) 5′-CGGGGTTAGAGGTTTTTGCGGTTGGA |
| linearly amplified templates | GGCTCATATCGAGGCTNNNNGTTCGCGAGAGT |
| Sequence in grey is the common | AGGCGCGT-3′ |
| backbone of all the unique | b) 5′-TCGGGTACGTGGTAGGTCGTGTTGGA |
| probes and “NNNN” represent PCR | GGCTCATATCGAGGCTNNNNTAGGCGGAAGTT |
| clone identifier. Shown here are | GGGAAGGCG-3′ |
| 2 padlock probes as an example. | |
| Repeat regions probes—targeting | a) LINE-padlock: |
| the bisulfite converted templates | AAACCTACCATTACTAAAACTTAAATAAACAT |
| | TGCGTGCACGTGGTCTCGACTGGTCCCTAATA |
| | CTATACTTTTCCAATAATC |
| | b) ALU-padlock |
| | CTAACCTCAAATAATCCACCTACCATTGCGTG |
| | CACGTGGTCTCGACTGGTAACAATCTTACTCT |
| | ATTACCTAAACTA |

TABLE 6
Universal primer pairs used
for multiplex PCR amplification
Universal Primer Pair

| | |
|---|---|
| NGS—Universal For (Next | 5′-CACCGAGATCTACACCACTC |
| generation sequencing | TCAGATGTTATCGAGGTCCGACA |
| (NGS)) | GGCTCATATCGAGGCT-3′ |
| NGS-Barcoded Universal | 5′-CAAGCAGAAGACGGCATACG |
| Rev (highlighted grey | AGATCGTGATGTGACTGGAGTTC |
| sequence is the bar | CGATATGAGCCTCCAAC-3′ |
| code—shown are 2 | 5′-CAAGCAGAAGACGGCATACG |
| primers with different | AGATACATCGGTGACTGGAGTTC |
| bar codes) | CGATATGAGCCTCCAAC-3′ |
| Universal_repeat-For | 5′-TGCCTAGCACGTGGTCTCGA |
| | CTGGT-3′ |
| Universal_repeat-Rev | 5′-GAGCTTCGACCACGTGCACG |
| | CAATG-3′ |

TABLE 7
Example Promoter regions (Human Genome hg19)
used for Method Validation

| Number | Gene | Chromosome | Start | End |
|---|---|---|---|---|
| 1 | APC | chr5 | 112737159 | 1127378585 |
| 2 | ARF1 | chr1 | 228082450 | 2280831495 |
| 3 | ARF1 | chr1 | 228081960 | 2280826595 |
| 4 | BCL2 | chr18 | 63319381 | 633200805 |
| 5 | BRCA1 | chr17 | 43125324 | 431260235 |
| 6 | CADM1 | chr11 | 115504524 | 1155052235 |
| 7 | CALCA | chr11 | 14972287 | 149729865 |
| 8 | CAV1 | chr7 | 116525658 | 1165263575 |
| 9 | CAV1 | chr7 | 116525593 | 1165262925 |
| 10 | CCND2 | chr12 | 4273036 | 42737355 |
| 11 | CDH1 | chr16 | 68736590 | 687372895 |
| 12 | CDKN2A | chr9 | 21994492 | 219951915 |
| 13 | CDKN2A | chr9 | 21974828 | 219755275 |
| 14 | CDKN2A | chr9 | 21975134 | 219758335 |
| 15 | CHFR | chr12 | 132887619 | 1328883185 |
| 16 | CYP1B1 | chr2 | 38076182 | 380768815 |
| 17 | DAPK1 | chr9 | 87497835 | 874985345 |
| 18 | DLC1 | chr8 | 13276549 | 132772485 |
| 19 | DLC1 | chr8 | 13133301 | 131340005 |
| 20 | DOK1 | chr2 | 74548320 | 745490195 |
| 21 | DOK1 | chr2 | 74553460 | 745541595 |
| 22 | EDNRB | chr13 | 77918832 | 779195315 |
| 23 | ESR1 | chr6 | 151806619 | 1518073185 |
| 24 | FHIT | chr3 | 61251462 | 612521615 |
| 25 | HIC1 | chr17 | 2055610 | 20563095 |
| 26 | HIC1 | chr17 | 2054399 | 20550985 |
| 27 | HOXA9 | chr7 | 27165531 | 271662305 |
| 28 | HS3ST2 | chr16 | 22813839 | 228145385 |
| 29 | HS3ST2 | chr16 | 22813839 | 228145385 |
| 30 | HSD17B4 | chr5 | 119451743 | 1194524425 |
| 31 | HSPA2 | chr14 | 64539768 | 645404675 |
| 32 | IGFBP3 | chr7 | 45921273 | 459219725 |
| 33 | MGMT | chr10 | 129466484 | 1294671835 |
| 34 | MIR148A | chr7 | 25949987 | 259506865 |
| 35 | MLH1 | chr3 | 36992650 | 369933495 |
| 36 | MLH1 | chr3 | 36993077 | 369937765 |
| 37 | MYOD1 | chr11 | 17718863 | 177195625 |
| 38 | NDRG4 | chr16 | 58463390 | 584640895 |
| 39 | NDRG4 | chr16 | 58462945 | 584636445 |
| 40 | NDRG4 | chr16 | 58464122 | 584648215 |
| 41 | NEUROG1 | chr5 | 135535950 | 1355366495 |
| 42 | NKX2 | chr14 | 36520226 | 365209255 |
| 43 | NKX2 | chr14 | 36519699 | 365203985 |
| 44 | NPTX2 | chr7 | 98616585 | 986172845 |
| 45 | PENK | chr8 | 56446735 | 564474345 |
| 46 | PGR | chr11 | 101129064 | 1011297635 |
| 47 | POU4F2 | chr4 | 146638193 | 1466388925 |
| 48 | PPP1R13B | chr14 | 103847591 | 1038482905 |
| 49 | PTEN | chr10 | 87862738 | 878634375 |
| 50 | RARB | chr3 | 25427563 | 254282625 |
| 51 | RASSF1 | chr3 | 50340937 | 503416365 |
| 52 | RASSF1 | chr3 | 50337465 | 503381645 |
| 53 | RUNX3 | chr1 | 24965011 | 249657105 |
| 54 | RUNX3 | chr1 | 24930280 | 249309795 |
| 55 | SCGB3A1 | chr5 | 180591488 | 1805921875 |
| 56 | SFRP1 | chr8 | 41309472 | 413101715 |
| 57 | SFRP2 | chr4 | 153789077 | 1537897765 |
| 58 | SFRP5 | chr10 | 97772000 | 977726995 |
| 59 | SHOX2 | chr3 | 158106164 | 1581068635 |
| 60 | SOCS1 | chr16 | 11256183 | 112568825 |
| 61 | SPET9 | chr17 | 77287191 | 772878905 |
| 62 | SPET9 | chr17 | 77280710 | 772814095 |
| 63 | SPET9 | chr17 | 77372490 | 773731895 |
| 64 | SPET9 | chr17 | 77449831 | 774505305 |
| 65 | SPET9 | chr17 | 77287191 | 772878905 |
| 66 | SYK | chr9 | 90801227 | 908019265 |
| 67 | SYK | chr9 | 90800980 | 908016795 |
| 68 | TERT | chr5 | 1295048 | 12957475 |
| 69 | THBS1 | chr15 | 39580379 | 395810785 |
| 70 | TMEFF2 | chr2 | 192194934 | 1921956335 |
| 77 | TNFRSF25 | chr1 | 6466196 | 64668955 |
| 78 | WRN | chr8 | 31032562 | 310332615 |
| 79 | ZNF154 | chr19 | 57709212 | 577099115 |

TABLE 8
Locus-specific real-time primer pairs

| Locus ID | Primer name | Primer Sequence |
|---|---|---|
| Locus_25 | Rev_CDKN2A_ori_25 | 5′-GTACAACGATTTAATTTAATTTCGCT-3′ |
| | For_CDKN2A_ori_25 | 5′-CGAGGTTATTTTATTGTTTTATTCGT-3′ |
| Locus_9 | Rev_BRCA1_ori_9 | 5′-CCCTAATAAAAATCTCCAATTTCGA-3′ |
| | For_BRCA1_ori_9 | 5′-TATTGTGGCGAAGATTTTTTATTTCG-3′ |
| Locus_5 | Rev_ARF1_ori_5 | 5′-TAAACCACAAACTATCTTCGCGA-3′ |
| | For_ARF1_ori_5 | 5′-GTCGGGGATATTTTGTTTCGG-3′ |
| Locus_2 | Rev_ARF1_ori_2 | 5′-ACCCTACCCCGAACCGC-3′ |
| | For_ARF1_ori_2 | 5′-ACGTTAAACGGGCGGGAGT-3′ |
| Locus_136 | Rev_SPET9_ori_136 | 5′-CCTTCCCCGAACGCAAAATC-3′ |
| | For_SPET9_ori_136 | 5′-TTTTGTTTGTTAGTCGCGTGCGT-3′ |
| Locus_133 | Rev_SPET9_ori_133 | 5′-CCTCCTCGCCATAACCCG-3′ |
| | For_SPET9_ori_133 | 5′-AGGCGAGAGACGCGGTTTTA-3′ |
| Locus_23 | Rev_CDKN2A_ori_23 | 5′-TACTAACAAACGAAAAAACGCGACT-3′ |
| | For_CDKN2A_ori_23 | 5′-GTATTAGTCGGAAGTAGTTTTCGT-3′ |
| Locus_21 | Rev_CDKN2A_ori_21 | 5′-CGAAATTAATAACACCTCCTCCGA-3′ |
| | For_CDKN2A_ori_21 | 5′-GTTGGCGGAAGAGTTTTTTTCGA-3′ |
| Locus_8 | Rev_BRCA1_ori_8 | 5′-ACCACGAAAACCAAAAAACTACCG-3′ |
| | For_BRCA1_ori_8 | 5′-GGGTGGTTAATTTAGAGTTTCGAG-3′ |
| Locus_113 | Rev_RASSF1_ori_113 | 5′-AACTTACAATCTACAAAAAAACCTAACGA-3′ |
| | For_RASSF1_ori_113 | 5′-GGAGTTTGGCGAGTAGCGGT-3′ |

### PARTS LIST

50—DNA obtaining step
52—gDNA purification step
54—bisulfite conversion step
56—optional repair step
58—linear amplification
60—optional cleaning step
62—optional genomic region capture step
64—Multiplex PCR step
66—verification step
68—template improvement step
70—genomic mapping analysis
111—complimentary template
113—linear amplification primer
115—extension and ligation step
117—exonuclease step
119—USER step
121—methylated fragments
123—non-annealed probes
125—linearly amplified fragment
200—multiplex probes

1.-22. (canceled)

23. A method for analysis of a sample nucleic acid sequence containing methylated cytosine, comprising:
providing a sample of nucleic acid sequences;
chemical treatment of said sample nucleic acid sequences resulting in a conversion of unmethylated cytosine residues in said sample nucleic acid sequence to uracil;
linearly amplifying said chemically treated nucleic acid sequence to generate a complimentary template to the chemically treated nucleic acid sequence, using at least one primer to target and overlap a region of interest of the genomic nucleic acid sequence; and
amplifying the complementary template via multiplex polymerase chain reaction (PCR) to generate a library of amplified nucleic acid sequences;
wherein the library of amplified nucleic acid sequences preferentially contain sequences from the sample nucleic acid that contained the region of interest,
wherein the at least one primer comprises a CpG dinucleotide for preferential amplification of methylated fragments of the region of interest or a TpG nucleotide for preferential amplification of unmethylated fragments of the region of interest.

24. A method for analysis of a sample nucleic acid sequence containing methylated cytosine, comprising:
providing a sample of nucleic acid sequences;
chemical treatment of said sample nucleic acid sequences resulting in a conversion of unmethylated cytosine residues in said sample nucleic acid sequence to uracil;
linearly amplifying said chemically treated nucleic acid sequence to generate a complimentary template to a portion of the chemically treated nucleic acid sequence, using at least one primer to target and overlap a region of interest of the genomic nucleic acid sequence;
contacting the sample with a plurality of nucleic acid probes, wherein the probes are designed to hybridize randomly along a target nucleic acid sequence;
allowing hybridization of the plurality of nucleic add probes to the target nucleic acid sequence;
forming a plurality of circular nucleic add sequences, each of the circular sequences comprising a nucleic acid probe sequence and a target nucleic acid sequence;
amplifying the plurality of circular nucleic acid sequences to form a plurality of amplified target nucleic add sequences; and
optionally, sequencing the amplified target nucleic add sequences,
wherein the plurality of amplified nucleic acid sequences preferentially contain sequences from the sample nucleic acid that contained the region of interest,
wherein the at least one primer comprises a CpG dinucleotide for preferential amplification of methylated fragments of the region of interest or a TpG nucleotide for preferential amplification of unmethylated fragments of the region of interest;
wherein the conversion of cytosine residues further comprises converting unmethylated cytosine residues, wherein 5-methyl-cytosine residues remain unchanged; and
wherein the chemical treatment is a bisulfite treatment.

25. A method for analysis of a sample nucleic acid sequence containing methylated cytosine, comprising:
providing a sample of nucleic acid sequences;
chemical treatment of said sample nucleic acid sequences resulting in a conversion of unmethylated cytosine residues in said sample nucleic acid sequence to uracil;
linearly amplifying said chemically treated nucleic acid sequence to generate a complimentary template to a portion of the chemically treated nucleic acid sequence, using at least one primer to target and overlap a region of interest of the genomic nucleic acid sequence; and
performing a two tier linear amplification to generate a library of amplified nucleic acid sequences;
wherein the library of amplified nucleic acid sequences preferentially contain sequences from the sample nucleic acid that contained the region of interest,
wherein the at least one primer comprises a CpG dinucleotide for preferential amplification of methylated fragments of the region of interest or a TpG nucleotide for preferential amplification of unmethylated fragments of the region of interest.

26. The method of claim 23 further comprising a repair step wherein DNA fragments are annealed together, prior to the bisulfite treatment followed by the linear amplification.

27. The method of claim 23 further comprising cleaving the chemically treated nucleic acid sequence at uracil residues utilizing a uracil DNA glycosylase enzyme, after the linear amplification step.

28. The method of claim 23 wherein the sample nucleic acid sequence is genomic DNA, preferably whole genomic DNA, for example, genomic DNA isolated from a blood sample from a patient.

29. The method of claim 23, further comprising using at least two primers during the multiplex PCR step.

30. The method of claim 23, wherein the multiplex PCR comprises two to twenty-two cycles.

31. The method of claim 23, wherein the probes are designed to hybridize to promoter regions along a target nucleic acid sequence.

32. The method of claim 23, wherein amplification primers hybridize to nucleic acid probe sequences during the multiplex amplification step.

33. The method of claim 23, wherein the nucleic acid probes are padlock probes.

34. The method of claim 23, wherein the target nucleic acid sequence, or the region of interest, is a gene or a promoter region.

35. The method of claim 24 wherein the differences in methylation pattern of the region of interest are known to correlate to a disease state such as cancer.

36. The method of claim 23 further comprising a genomic region capture step, prior to the multiplex amplification step.

37. A method of determining whether a patient has a cancer, comprising performing the method of claim 23 to a sample from the patient, and comparing the amount of amplified DNA from the method to a control sample, wherein a higher amount of amplified DNA is determinative of cancer.


SEQUENCE LISTING (55 sequences)

SEQ ID NO: 1 - DNA, 16 bp, Artificial
  primer
aaacaccctaccccga

SEQ ID NO: 2 - DNA, 16 bp, artificial
  Primer
tttccgttaccacgaa

SEQ ID NO: 3 - DNA, 16 bp, ARTIFICIAL
  PRIMER
tcccccactctttccg

SEQ ID NO: 4 - DNA, 16 bp, artificial
  primer
cttcccaccctcaacg

SEQ ID NO: 5 - DNA, 18 bp, artificial
  primer
cattcgctaaatactcga

SEQ ID NO: 6 - DNA, 17 bp, artificial
  primer
gactctaaaccctacgc

SEQ ID NO: 7 - DNA, 17 bp, artificial
  primer
ccaaacaaacgaacgcg

SEQ ID NO: 8 - DNA, 18 bp, artificial
  primer
ctacaaaaaaaccctacg

SEQ ID NO: 9 - DNA, 14 bp, artificial
  primer
ccttccccgaacgc

SEQ ID NO: 10 - DNA, 21 bp, artificial
  primer
  uracil (1)..(20): n is uracil
  misc_feature (21)..(21): n is uracil
tgcctagggtctcgactggcn

SEQ ID NO: 11 - DNA, 20 bp, artificial
  primer
gagcttcggtgcacgcaatg

SEQ ID NO: 12 - DNA, 20 bp, artificial
  primer
accagtcgagaccctaggca

SEQ ID NO: 13 - DNA, 20 bp, artificial
  primer
  misc_feature (20)..(20): n is uracil
tgcctaggctgagcagtgcn

SEQ ID NO: 14 - DNA, 20 bp, artificial
  primer
gagcttcgcacgtggcaatg

SEQ ID NO: 15 - DNA, 20 bp, artificial
  primer
agcactgctcagcctaggca

SEQ ID NO: 16 - DNA, 66 bp, artificial
  primer
  misc_feature (43)..(46): n is a, c, g, or t
cggggttagaggtttttgcggttggaggctcatatcgaggctnnnngttcgcgagagtaggcgcgt

SEQ ID NO: 17 - DNA, 67 bp, artificial
  primer
  misc_feature (43)..(46): n is a, c, g, or t
tcgggtacgtggtaggtcgtgttggaggctcatatcgaggctnnnntaggcggaagttgggaaggcg

SEQ ID NO: 18 - DNA, 83 bp, artificial
  primer
aaacctaccattactaaaacttaaataaacattgcgtgcacgtggtctcgactggtccctaatactatacttttccaataatc

SEQ ID NO: 19 - DNA, 77 bp, artificial
  primer
ctaacctcaaataatccacctaccattgcgtgcacgtggtctcgactggtaacaatcttactctattacctaaacta

SEQ ID NO: 20 - DNA, 59 bp, artificial
  primer
caccgagatctacaccactctcagatgttatcgaggtccgacaggctcatatcgaggct

SEQ ID NO: 21 - DNA, 60 bp, artificial
  primer
caagcagaagacggcatacgagatcgtgatgtgactggagttccgatatgagcctccaac

SEQ ID NO: 22 - DNA, 60 bp, artificial
  primer
caagcagaagacggcatacgagatacatcggtgactggagttccgatatgagcctccaac

SEQ ID NO: 23 - DNA, 25 bp, artificial
  primer
tgcctagcacgtggtctcgactggt

SEQ ID NO: 24 - DNA, 25 bp, artificial
  primer
gagcttcgaccacgtgcacgcaatg

SEQ ID NO: 25 - DNA, 26 bp, artificial
  primer
gtacaacgatttaatttaatttcgct

SEQ ID NO: 26 - DNA, 26 bp, artificial
  primer
cgaggttattttattgttttattcgt

SEQ ID NO: 27 - DNA, 25 bp, artificial
  primer
ccctaataaaaatctccaatttcga

SEQ ID NO: 28 - DNA, 26 bp, artificial
  primer
tattgtggcgaagattttttatttcg

SEQ ID NO: 29 - DNA, 23 bp, artificial
  primer
taaaccacaaactatcttcgcga

SEQ ID NO: 30 - DNA, 21 bp, artificial
  primer
gtcggggatattttgtttcgg

SEQ ID NO: 31 - DNA, 17 bp, artificial
  primer
accctaccccgaaccgc

SEQ ID NO: 32 - DNA, 19 bp, artificial
  primer
acgttaaacgggcgggagt

SEQ ID NO: 33 - DNA, 20 bp, artificial
  primer
ccttccccgaacgcaaaatc

SEQ ID NO: 34 - DNA, 23 bp, artificial
  primer
ttttgtttgttagtcgcgtgcgt

SEQ ID NO: 35 - DNA, 18 bp, artificial
  primer
cctcctcgccataacccg

SEQ ID NO: 36 - DNA, 20 bp, artificial
  primer
aggcgagagacgcggtttta

SEQ ID NO: 37 - DNA, 25 bp, artificial
  primer
tactaacaaacgaaaaaacgcgact

SEQ ID NO: 38 - DNA, 24 bp, artificial
  primer
gtattagtcggaagtagttttcgt

SEQ ID NO: 39 - DNA, 24 bp, artificial
  primer
cgaaattaataacacctcctccga

SEQ ID NO: 40 - DNA, 23 bp, artificial
  primer
gttggcggaagagtttttttcga

SEQ ID NO: 41 - DNA, 24 bp, artificial
  primer
accacgaaaaccaaaaaactaccg

SEQ ID NO: 42 - DNA, 24 bp, artificial
  primer
gggtggttaatttagagtttcgag

SEQ ID NO: 43 - DNA, 29 bp, artificial
  primer
aacttacaatctacaaaaaaacctaacga

SEQ ID NO: 44 - DNA, 20 bp, artificial
  primer
ggagtttggcgagtagcggt

SEQ ID NO: 45 - DNA, 16 bp, human
accccgacgatcacgt

SEQ ID NO: 46 - DNA, 16 bp, human
  misc_feature (2)..(5): n is uracil
  misc_feature (8)..(8): n is uracil
  misc_feature (12)..(12): n is uracil
  misc_feature (14)..(14): n is uracil
annnngangatnangt

SEQ ID NO: 47 - DNA, 16 bp, human
attttgatgattatgt

SEQ ID NO: 48 - DNA, 16 bp, human
taaaactactaataca

SEQ ID NO: 49 - DNA, 16 bp, human
accccgacgatcacgt

SEQ ID NO: 50 - DNA, 16 bp, human
  misc_feature (2)..(4): n is uracil
  misc_feature (12)..(12): n is uracil
annncgacgatnacgt

SEQ ID NO: 51 - DNA, 16 bp, human
atttcgacgattacgt

SEQ ID NO: 52 - DNA, 16 bp, human
taaagctgctaatgca

SEQ ID NO: 53 - DNA, 32 bp, human
  misc_feature (2)..(4): n is uracil
  misc_feature (12)..(12): n is uracil
  misc_feature (18)..(20): n is uracil
  misc_feature (23)..(23): n is uracil
  misc_feature (28)..(28): n is uracil
annncgacgatnacgtannncgncgatnacgt

SEQ ID NO: 54 - DNA, 32 bp, human
taaagctgctaatgcataaagcagctaatgca

SEQ ID NO: 55 - DNA, 19 bp, human
  misc_feature (2)..(4): n is uracil
  misc_feature (12)..(12): n is uracil
  misc_feature (18)..(19): n is uracil
annncgacgatnacgtann